(19) United States
(12) Patent Application Publication
(10) Pub. No.: US 2007/0218551 A1
(43) Pub. Date: Sep. 20, 2007

(54) Novel Sirna-Based Approach to Target the Hif-Alpha Factor for Gene Therapy

(76) Inventors: Chuan-Yuan Li, Chapel Hill, NC (US); Xiuwu Zhang, Mornsville, NC (US); Mark W. Dewhirst, Durham, NC (US)

Correspondence Address: JENKINS, WILSON, TAYLOR & HUNT, P. A., SUITE 1200, UNIVERSITY TOWER, 3100 TOWER BOULEVARD, DURHAM, NC, 27707, US

(21) Appl. No.: 10/574,129

(22) PCT Filed: Oct. 4, 2004
(86) PCT No.: PCT/US04/32710

§ 371 Date: Nov. 2, 2006

Related U.S. Application Data
(60) Provisional application No. 60/508,145, filed on Oct. 2, 2003.

(51) Int. Cl. C12N 15/861; C07H 21/02; C12N 5/08; C12N 15/63

(52) U.S. Cl. 435/366; 435/320.1; 435/375; 536/24.5

(57) ABSTRACT

The presently disclosed subject matter provides vectors that encode small interfering RNAs (siRNAs) designed to down regulate the expression of hypoxia-inducible genes in a cell. Also provided are compositions and host cells based upon the vectors, as well as methods of using the vectors. The presently disclosed subject matter further provides a method of inhibiting tumor growth infecting cells in a tumor with an adenovirus vector that encodes an siRNA directed against the hypoxia-inducible factor 1α (HIF-1α) gene.

### CROSS REFERENCE TO RELATED APPLICATIONS

This application is based on and claims priority to U.S. Provisional Patent application Ser. No. 60/508,145, filed Oct. 2, 2003, herein incorporated by reference in its entirety.

### GRANT STATEMENT

This work was supported by grant CA81512 from the U.S. National Institutes of Health (NIH). Thus, the U.S. government has certain rights in the presently disclosed subject matter.

### TECHNICAL FIELD

The presently disclosed subject matter generally relates to methods and compositions for inhibiting the expression of hypoxia-inducible genes in a hypoxic cell. More particularly, the methods and compositions involve contacting hypoxic cells, for example a hypoxic cell in a tumor, with a vector encoding a small interfering RNA (siRNA) such that the siRNA is expressed in the hypoxic cell, killing the cell.

### Table of Abbreviations

2′-H—2′-deoxy
2,5-A—2′,5′-linked oligoadenylates
5′-O-DMT—5′-terminal dimethoxytrityl
A—adenine
ACN—acrylonitrile
Ad—adenovirus
AdsiHIF-1α—an adenovirus vector encoding an siRNA directed against HIF-1α
AdsiNT—an adenovirus vector encoding a control siRNA with no known homology to any target gene (i.e. a non-targeted siRNA)
ARNT—aryl hydrocarbon receptor nuclear translocator
ATCC—American Type Culture Collection
C—cytosine
CAT—chloramphenicol acetyltransferase
CMV—cytomegalovirus
CV—column volume
DHFR—dihydrofolate reductase
DIPA—diisopropylethylamine
DMAP—dimethylaminopurine
DMEM—Dulbecco's modified Eagle's medium
DMSO—dimethylsulfoxide
dsRNA—double stranded RNA
EDTA—ethylenediamine tetraacetic acid
FBS—fetal bovine serum
FLT-1—a receptor for VEGF
G—guanine
GFP—green fluorescent protein
HF—hydrogen fluoride
HIF-1—hypoxia-inducible factor 1
HIF-1α—hypoxia-inducible factor 1α
HIF-1β—hypoxia-inducible factor 1; ARNT
HPLC—high performance liquid chromatography
HPRT—hypoxanthine phosphoribosyl transferase
HREs—hypoxia responsive elements
HRP—horseradish peroxidase
hsp—heat shock protein
IFN-α—interferon alpha
IFN-γ—interferon gamma
IgG—immunoglobulin gamma
IL2—interleukin 2
IL4—interleukin 4
IL6—interleukin 6
m.o.i.—multiplicity of infection
NaOAc—sodium acetate
NIH—National Institutes of Health
PAGE—polyacrylamide gel electrophoresis
PBS—phosphate-buffered saline
PBST—phosphate-buffered saline plus Tween 20
pfu—plaque-forming unit
PKR—RNA-dependent protein kinase
PSA—prostate serum antigen
PyBrOP—bromotripyrrolidinophosphoniumhexafluororophosphate
pVHL—von Hippel-Lindau protein
RISC—RNA-induced silencing complex
RNAi—RNA interference
SDS—sodium dodecyl sulfate
SE—standard error
siRNA—small (or short) interfering RNA
SV40—simian virus 40
   SSC—standard saline citrate
   T—thymine
   TAFs—Transcription Associated Factors
   TCA—trichloroacetate
   TEA—triethylamine
   TEM—triethylamine acetate
   TFA—trifluoroacetic acid
   THF—tetrahydrofuran
   Tₘ—thermal melting point
   TNF—tumor necrosis factor
   U—uracil
   VEGF—vascular endothelial growth factor

### BACKGROUND

Despite significant advances in medical research and technology, cancer continues to be one of the leading causes of death in the United States and throughout the world. There are in excess of one million new cases of cancer reported in the United States alone, and more than half a million people die in this country every year from cancer.

Current treatments for cancer include surgical removal and/or radiation treatment of tumors, yet each has its limitations. In the former case, once a tumor has metastasized by invading the surrounding tissue or by moving to a distant site, it can be virtually impossible for the surgeon to remove all cancerous cells. Any such cells left behind can continue growing, leading to a recurrence of cancer following surgery. Current radiation therapy strategies are also frequently unsuccessful at eradicating a patient's cancer. Following radiation therapy, cancer can recur because it is often not possible to deliver a sufficiently high dose of radiation to kill all the tumor cells without at the same time injuring the surrounding normal tissue. Cancer can also recur because tumors show widely varying susceptibilities to radiation-induced cell death. Thus, the inability of current treatment protocols to eliminate tumor cells is an important clinical limitation leading to unsuccessful cancer therapy (Lindegaard et al., 1996; Suit, 1996; Valter et al., 1999).

Newer treatment strategies are needed to address the challenges that result from the inability to successfully treat neoplastic disease. One of the major challenges facing the medical oncologist is selectivity: the ability to kill tumor cells without causing damage to normal cells in the surrounding area. Various current approaches take advantage of the fact that in most cases tumor cells grow more quickly than normal cells, so strategies designed to kill rapidly growing cells are somewhat selective for tumor cells (see Yazawa et al., 2002). However, these methods also kill certain cell types in the body that normally divide rapidly, most notably cells in the bone marrow, resulting in complications such as anemia and neutropenia (reviewed in Vose & Armitage, 1995). Other strategies are based upon the production of antibodies directed against tumor-specific antigens (reviewed in Sinkovics & Horvath, 2000). However, few such antigens have been identified, limiting the applicability of these approaches. Thus, there is a need for new methods to enhance the selectivity of cancer treatment approaches.

Hypoxia, a state of lower than normal tissue oxygen tension, has recently been implicated in a host of human diseases, including cancer. It is prominently involved in tumor growth and development. Specifically, hypoxia is found to play a critical role in promoting mutagenesis and selecting for malignant tumor cells. It is also involved in promoting tumor angiogenesis.

Cellular responses to hypoxia are primarily mediated by the transcription factor hypoxia inducible factor 1 (HIF-1). Under conditions of low oxygen, HIF-1 binds to sequences called hypoxia responsive elements (HREs) that are present in the promoters of certain hypoxia responsive genes. The binding of HIF-1 to an HRE-containing promoter results in up-regulated transcription of the associated gene.

The active form of HIF-1 is a heterodimer composed of a regulatory component (HIF-1α) and the constitutively expressed aryl hydrocarbon receptor nuclear translocator (ARNT, also called HIF-1β). The regulation of HIF-1-mediated transcription occurs via post-translational modifications of HIF-1α that depend upon the oxygen status of the cell. Under normoxic conditions, HIF-1α is hydroxylated by the enzyme prolyl hydroxylase using molecular oxygen as the oxygen donor. This hydroxylation allows von Hippel-Lindau protein (pVHL), which is normally present within the cell, to bind to HIF-1α, forming a pVHL/HIF-1α complex. The pVHL/HIF-1α complex is subject to ubiquitylation and degradation in the proteasome. Under hypoxic conditions, on the other hand, prolyl hydroxylase activity is much lower due to the relative absence of the oxygen donor. Under these conditions, HIF-1α is not hydroxylated, pVHL/HIF-1α complexes do not form, and the steady state level of HIF-1α within the cell increases. HIF-1α is thus available to form active HIF-1 by complexing with HIF-1β, which results in the transcription of those genes with HRE-containing promoters.

HIF-1 binding results in increased expression of several genes, including transcription factors, growth factors, and cytokines, as well as genes involved in oxygen transport and iron metabolism, glycolysis and glucose uptake, and stress-response. In addition, hypoxia regulates cellular proliferation and migration related to angiogenesis. The vascular endothelial growth factor (VEGF) gene, the product of which is a critical regulatory factor in angiogenesis, contains an HRE in its promoter. HIF-1 upregulates the expression of VEGF and FLT-1, a VEGF receptor. Due to the high growth rate of the cells that make up a solid tumor, new blood vessels are constantly needed to provide rapidly growing tumor cells with adequate nutrients, including oxygen. These newly formed blood vessels frequently are characterized by abnormalities, such that it is very common to find areas of tumors in which individual cells fail to be oxygenated sufficiently. In fact, published data suggest that there are localized regions of hypoxia in virtually every tumor larger than 1 mm³ (Dachs & Tozer, 2000).

Given the primary role HIF-1 plays in cellular responses to hypoxia and the presence of hypoxic regions in solid tumors, it might be possible to exploit the mechanisms cells use to respond to hypoxia as points of entry for therapeutic intervention. On a biochemical level, it might be possible to prevent the changes tumor cells undergo under hypoxic conditions by interfering with the cascade of gene expression that is regulated by HIF-1. What is needed, therefore, is an efficient way to prevent the accumulation of activated HIF-1 in a cell, such that when the cell is exposed to hypoxia it is unable to adapt to low oxygen tension and thus undergoes apoptosis.

Thus, there exists a long-felt and continuing need in the art for effective therapies to specifically target and kill tumor cells in a subject. The presently disclosed subject matter addresses this and other needs in the art.

### SUMMARY

The presently disclosed subject matter provides methods for inhibiting the expression of a hypoxia-inducible gene in a cell in hypoxic conditions or expected to undergo hypoxic conditions. In some embodiments, the method comprises introducing a ribonucleic acid (RNA) into the cell in an amount sufficient to inhibit expression of the hypoxia-inducible gene, wherein the RNA comprises a ribonucleotide sequence that corresponds to a coding strand of the hypoxia-inducible gene. In some embodiments, the hypoxia-inducible gene is HIF-1α. In some embodiments, the HIF-1α gene comprises a nucleotide sequence of one of SEQ ID NOs: 1 and 3.

In some embodiments of the present method, the RNA comprises a double-stranded region comprising a first strand comprising a ribonucleotide sequence that corresponds to a coding strand of the hypoxia-inducible gene and a second strand comprising a ribonucleotide sequence that is complementary to the first strand, and wherein the first strand and the second strand hybridize to each other to form the double-stranded molecule. In some embodiments, the double stranded region is at least 15 basepairs in length. In some embodiments, the double stranded region is between 15 and 50 basepairs in length. In some embodiments, the double stranded region is between 19 and 30 basepairs in length. In some embodiments, the length of the double stranded region is selected from the group consisting of 19, 20, 21, 22, 23, 24, 25, 26, 27, 28, 29, and 30 basepairs.

In some embodiments of the present method, the expression of the hypoxia-inducible gene is inhibited by at least 10%.

In some embodiments of the present method, the RNA comprises one strand that forms a double-stranded region by intramolecular self-hybridization that is complementary over at least 19 bases. In some embodiments of the present method, the RNA comprises two separate strands that form a double-stranded region by intermolecular hybridization that is complementary over at least 19 bases.

The present method can be used to inhibit the expression of a hypoxia-inducible gene in a cell present in an organism. In some embodiments, the RNA is introduced into the organism. In some embodiments, the RNA is introduced by extracellular injection into the organism.

In some embodiments of the present method, the method further comprises introducing a vector into the cell, wherein the vector encodes the RNA.

The presently disclosed subject matter also provides a method for inhibiting expression of a hypoxia-inducible gene in a subject. In some embodiments, the method comprises (a) providing a subject containing a target cell, wherein the target cell comprises the hypoxia-inducible gene and the hypoxia-inducible gene is expressed in the target cell when the target cell is exposed to hypoxic conditions; and (b) introducing a small interfering RNA (siRNA) into the target cell, wherein the siRNA comprises a nucleic acid sequence corresponding to the hypoxia-inducible gene. In some embodiments, the subject is an animal. In some embodiments, the small interfering RNA (siRNA) comprises a double-stranded structure with duplexed ribonucleic acid strands and one of the strands is complementary to a portion of the hypoxia-inducible gene. In some embodiments, the small interfering RNA (siRNA) is introduced into the subject and outside the target cell. In some embodiments, the small interfering RNA (siRNA) is introduced into the target cell by introducing a vector encoding the small interfering RNA (siRNA) into the target cell.

The presently disclosed subject matter also provides a method for suppressing the growth of a hypoxic cell in a subject, the method comprising contacting the cell with a vector comprising a small interfering RNA (siRNA) molecule under conditions sufficient to allow entry of the vector into the cell, wherein the siRNA molecule comprises a sense region and an antisense region and wherein the antisense region comprises a nucleic acid sequence complementary to an RNA sequence encoding a hypoxia-inducible gene product and the sense region comprises a nucleic acid sequence complementary to the antisense region. In some embodiments of the present method, the cell is a tumor cell. In some embodiments, the tumor cell is in a hypoxic region of a tumor. In some embodiments, the subject is a mammal. In some embodiments, the vector comprises a liposome. In some embodiments, the hypoxia-inducible gene is hypoxia inducible factor 1 alpha (HIF-1α). In some embodiments, the HIF-1α gene comprises a nucleotide sequence of one of SEQ ID NOs: 1 and 3.

In some embodiments of the present method, the vector is introduced into the subject via a route of administration selected from the group consisting of intravenous administration, intrasynovial administration, transdermal administration, intramuscular administration, subcutaneous administration, topical administration, rectal administration, intravaginal administration, intratumoral administration, oral administration, buccal administration, nasal administration, parenteral administration, inhalation, and insufflation.

The presently disclosed subject matter also provides a method for suppressing the growth of a hypoxic cell in a subject. In some embodiments, the method comprises contacting the cell with a vector encoding a small interfering RNA (siRNA) molecule under conditions sufficient to allow entry of the vector into the cell, wherein the siRNA molecule comprises a sense region and an antisense region and wherein the antisense region comprises a first nucleic acid sequence that is 100% complementary to at least 19 contiguous nucleotides of a hypoxia-inducible gene sequence and the sense region comprises a second nucleic acid sequence that is 100% complementary to the first nucleic acid sequence. In some embodiments, the vector is an adenovirus vector. In some embodiments, the cell is a tumor cell. In some embodiments, the tumor cell is in a hypoxic region of a tumor. In some embodiments, the subject is a mammal. In some embodiments, the vector is an adenovirus vector. In some embodiments, the hypoxia-inducible gene is hypoxia inducible factor 1 alpha (HIF-1α). In some embodiments, the HIF-1α gene comprises a nucleotide sequence of one of SEQ ID NOs: 1 and 3. In some embodiments of the present method, the vector is introduced into the subject via a route of administration selected from the group consisting of intravenous administration, intrasynovial administration, transdermal administration, intramuscular administration, subcutaneous administration, topical administration, rectal administration, intravaginal administration, intratumoral administration, oral administration, buccal administration, nasal administration, parenteral administration, inhalation, and insufflation.

The presently disclosed subject matter also provides a small interfering RNA (siRNA) molecule that down regulates expression of a hypoxia-inducible factor 1α (HIF-1α) gene by RNA interference. In some embodiments, the siRNA molecule comprises a sense region and an antisense region and wherein the antisense region comprises a first nucleic acid sequence that is 100% complementary to at least 10 contiguous nucleotides of a hypoxia-inducible factor 1α (HIF-1α) gene sequence and the sense region comprises a second nucleic acid sequence that is 100% complementary to the first nucleic acid sequence. In some embodiments, the siRNA molecule is assembled from two nucleic acid fragments, wherein one fragment comprises a sense region and the other fragment comprises an antisense region of the siRNA molecule. In some embodiments, the sense region and antisense region are covalently connected via a linker molecule. In some embodiments, the linker molecule is a polynucleotide linker. In some embodiments, the polynucleotide linker comprises from 5 to 9 nucleotides. In some embodiments, the linker molecule is a non-nucleotide linker.

In some embodiments, the sense region comprises a 19-30 base sequence selected from SEQ ID NOs. 1 and 3.

In some embodiments of the present method, one or both of the sense region and antisense regions comprises a 3′-terminal overhang. In some embodiments, a 3′-terminal overhang comprises from 2 to 8 nucleotides. In some embodiments, the antisense region 3′-terminal nucleotide overhang is complementary to a ribonucleic acid (RNA) encoding hypoxia-inducible factor 1α (HIF-1α).

The presently disclosed subject matter also encompasses nucleic acid molecules having various modifications. In some embodiments, the sense region of the disclosed siRNA molecule comprises one or more modified pyrimidine nucleotides. In some embodiments, the sense region of the disclosed siRNA molecule comprises a terminal cap moiety at the 5′-end, the 3′-end, or combinations thereof. In some embodiments, the antisense region of the disclosed siRNA molecule comprises one or more modified pyrimidine nucleotides. In some embodiments, the antisense region of the disclosed siRNA molecule comprises a phosphorothioate internucleotide linkage at the 3′ end of the antisense region. In some embodiments, the antisense region of the disclosed siRNA molecule comprises 1-5 phosphorothioate internucleotide linkages at the 5′ end of the antisense region. In some embodiments, the 3′-terminal nucleotide overhang comprises one or more chemically modified nucleotides. In some embodiments, the 3′-terminal nucleotide overhang comprises ribonucleotides that are chemically modified at a nucleic acid sugar, base, or backbone position. In some embodiments, the 3′-terminal nucleotide overhang comprises one or more universal base ribonucleotides. In some embodiments, the 3′-terminal nucleotide overhang comprises one or more acyclic nucleotides.

The compositions of the presently disclosed subject matter can also be provided in a pharmaceutically acceptable carrier.

The presently disclosed subject matter also provides an expression vector comprising a nucleic acid sequence encoding at least one siRNA molecule as disclosed herein, as well as a mammalian cell comprising the disclosed expression vector. In some embodiments, the mammalian cell is a human cell. In some embodiments of the present expression vector, the siRNA molecule comprises a sense region and an antisense region and wherein the antisense region comprises a nucleic acid sequence complementary to an RNA sequence encoding a hypoxia-inducible factor 1α (HIF-1α) and the sense region comprises a nucleic acid sequence complementary to the antisense region. In some embodiments of the present expression vector, the siRNA molecule comprises two distinct strands having complementary sense and antisense regions. In some embodiments of the present expression vector, the siRNA molecule comprises a single strand having complementary sense and antisense regions.

Accordingly, it is an object of the presently disclosed subject matter to provide a method that employs an adenovirus vector to deliver an siRNA to a cell expressing hypoxia inducible factor 1 (HIF-1). This and other objects are achieved in whole or in part by the presently disclosed subject matter.

An object of the presently disclosed subject matter having been stated above, other objects and advantages of the presently disclosed subject matter will become apparent to those of ordinary skill in the art after a study of the following description and non-limiting Examples.

### BRIEF DESCRIPTION OF THE DRAWINGS

FIG. 1 depicts siRNA-mediated down regulation of HIF-1α expression. HeLa cells were infected with an adenovirus encoding an siRNA directed against HIF-1α (AdsiHIF-1α) or a negative control adenovirus (AdsiNT) for 24 hours and then subjected to hypoxia (0.5% O₂) for 24 hours. The cells were then harvested and analyzed by western blot. AdsiHIF-1α-infected cells showed more than 90% down regulation of HIF-1α expression.

FIGS. 2A and 2B depict the sensitization of HeLa cells to hypoxia-induced cell death by siRNA mediated HIF-1α down regulation.

FIG. 2A depicts apoptosis in hypoxic HeLa cells as evaluated by Hoechst 33342 staining. HeLa cells were infected with AdsiHIF-1α or AdsiNT vectors for 24 hours and subjected to hypoxia (0.5% O₂) for 24 hours. The cells were stained with then Hoechst 33342 dye. The nuclei of cells appeared intensely fluorescent, fragmented, and condensed, consistent with changes associated with apoptosis. The top panel depicts typical cells in each treatment condition while the lower panel represents the results of quantitative analyses. The size bar represents 25 μm.

FIG. 2B depicts molecular analysis of apoptotic protein expression. HeLa cells were infected with AdsiHIF-1α or AdsiNT vectors for 24 hours and exposed to hypoxia for 24 hours. Protein was then extracted and western blot analysis was performed using antibodies against the cleaved form of caspase-3 (17 kilodalton form), Bcl-X_{L}, and β-actin, the latter used as a control for protein loading. The lower panel depicts the results of densitometry analysis of the western blots.

FIGS. 3A-C depict the rate of tumor growth of cells transduced with an HIF-1α-targeted siRNA.

In FIGS. 3A and 3B, HeLa (FIG. 3A) or HCT116 (FIG. 3B) cells were first infected with either AdsiNT or AdsiHIF-1α at an m.o.i. of 10. Twenty-four hours after infection, about 5×10⁶ tumor cells were injected subcutaneously into the flanks of nude mice. There were six animals in each treatment group. The measurement of tumor sizes was conducted on subsequent days. The error bars show the standard deviation in each group at each data point.

FIG. 3C depicts photomicrographs showing reduced HIF-1α levels (as indicated by the dark stain that resulted from an HIF-1α antibody) of an AdsiHF-1α infected HCT116 tumor. The top two panels depict tumors infected with the control AdsiNT virus while the lower two panels depict tumors infected with the AdsiHIF-1α virus. The two panels on the right half of FIG. 3C show magnified views of subregions of the two panels on the left half of FIG. 3C. The scale bar represents 100 μm in the depicted photomicrographs.

FIG. 4 presents' data related to tumor growth delay as a result of combined radiation and AdsiHIF1-α treatment in established HCT116 tumors. HCT116 tumors were established by subcutaneous injection of 5×10⁶ cells in 50 μl PBS. When tumor diameters reach 6-8 mm, three doses of AdsiHIF1-α or AdsiNT were administered every other day. Irradiation was carried out 24 hours following every viral injection in the combined treatment group. Shown in the graph are the profiles of relative tumor volumes in the various treatment groups after the initial virus injection. The error bars represent standard error of the mean (SEM).

FIG. 5 depicts a general structure for an siRNA molecule of the presently disclosed subject matter. For the double-stranded molecule shown in FIG. 5, N can be any nucleotide, provided that in the loop structure identified as N₅₋₉, all 5-9 nucleotides remain in a single-stranded conformation. Similarly, N₂₋₈ can be any sequence of 2-8 nucleotides or modified nucleotides, provided that the nucleotides remain in a single-stranded conformation in the siRNA molecule.

### BRIEF DESCRIPTION OF THE SEQUENCE LISTING

SEQ ID NOs: 1 and 2 are nucleic acid and deduced amino acid sequences, respectively, corresponding to a human HIF-1α cDNA (GENBANK® Accession No. NM_{—}001530).

SEQ ID NOs: 3 and 4 are nucleic acid and deduced amino acid sequences, respectively, corresponding to a murine HIF-1α cDNA (GENBANK0 Accession No. AF003695), respectively.

SEQ ID NO: 5 is a generic sequence of an siRNA directed to human HIF-1α. The sequence comprises 19 nucleotides from a human HIF-1α cDNA (bases 528-546 of GENBANK® Accession No. NM_{—}001530), followed by from 5-9 nucleotides of random sequence, followed by the reverse-complement of bases 528-546 of GENBANK® Accession No. NM_{—}001530, followed by from 2-8 nucleotides that form a 3′ overhang.

SEQ ID NO: 6 is a specific embodiment of the generic sequence represented by SEQ ID NO: 5. This siRNA molecule was used to target human HIF-1α in HeLa cells (see the section entitled “Discussion of Examples 4-11” beginning on page 62).

SEQ ID NO: 7 is the nucleic acid sequence of a sense strand of an siRNA used to target human HIF-1α.

SEQ ID NO: 8 is the sequence of the negative control minigene present in pSilencer-siNT.

SEQ ID NOs: 9-12 are the sequences of various primers used in Quantitative PCR reactions. SEQ ID NOs: 9 and 10 were used to amplify the β-actin gene product and SEQ ID NOs: 11 and 12 were used to amplify the HIF-1α gene product.

### DETAILED DESCRIPTION

The presently disclosed subject matter generally relates to methods and compositions for suppressing or inhibiting the growth of a cell that expresses a hypoxia-inducible gene. In some embodiments, the methods involve infecting hypoxic cells, for example a hypoxic cell in a tumor, with an adenovirus vector encoding an siRNA such that the nucleic acid molecule encoded by the siRNA is expressed in the cell, expression of the hypoxia-inducible gene is inhibited, and the cell undergoes apoptosis.

I. General Considerations

The presently disclosed subject matter takes advantage of the ability of short, double stranded RNA molecules to cause the down regulation of cellular genes, a process referred to as RNA interference. As used herein, “RNA interference” (RNAi) refers to a process of sequence-specific post-transcriptional gene silencing mediated by a small interfering RNA (siRNA). See generally Fire et al., 1998. The process of post-transcriptional gene silencing is thought to be an evolutionarily conserved cellular defense mechanism that has evolved to prevent the expression of foreign genes (Fire, 1999).

RNAi might have evolved to protect cells and organisms against the production of double stranded RNA (dsRNA) molecules resulting from infection by certain viruses (particularly the double stranded RNA viruses or those viruses for which the life cycle includes a double stranded RNA intermediate) or the random integration of transposon elements into the host genome via a mechanism that specifically degrades single stranded RNA or viral genomic RNA homologous to the double stranded RNA species.

The presence of dsRNA in cells triggers various responses, one of which is RNAi. RNAi appears to be different from the interferon response to dsRNA, which results from dsRNA-mediated activation of an RNA-dependent protein kinase (PKR) and 2′,5′-oligoadenylate synthetase, resulting in non-specific cleavage of mRNA by ribonuclease L.

The presence of long dsRNAs in cells stimulates the activity of the enzyme Dicer, a ribonuclease III. Dicer catalyzes the degradation of dsRNA into short stretches of dsRNA referred to as small interfering RNAs (siRNA) (Bernstein et al., 2001). The small interfering RNAs that result from Dicer-mediated degradation are typically about 21-23 nucleotides in length and contain about 19 base pair duplexes. After degradation, the siRNA is incorporated into an endonuclease complex referred to as an RNA-induced silencing complex (RISC). The RISC is capable of mediating cleavage of single stranded RNA present within the cell that is complementary to the antisense strand of the siRNA duplex. According to Elbashir et al., cleavage of the target RNA occurs near the middle of the region of the single stranded RNA that is complementary to the antisense-strand of the siRNA duplex (Elbashir et al., 2001b).

RNAi has been described in several cell type and organisms. Fire et al., 1998 described RNAi in C. elegans. Wianny & Zernicka-Goetz, 1999 disclose RNAi mediated by dsRNA in mouse embryos. Hammond et al., 2000 were able to induce RNAi in Drosophila cells by transfecting dsRNA into these cells. Elbashir et al. (2001a) demonstrated the presence of RNAi in cultured mammalian cells including human embryonic kidney and HeLa cells by the introduction of duplexes of synthetic 21 nucleotide RNAs.

Experiments using Drosophila embryonic lysates revealed certain aspects of siRNA length, structure, chemical composition, and sequence that are involved in RNAi activity. See Elbashir et al., 2001c. In this assay, 21 nucleotide siRNA duplexes were most active when they contain 3′-overhangs of two nucleotides. Also, the position of the cleavage site in the target RNA was shown to be defined by the 5′-end of the siRNA guide sequence rather than the 3′-end (Elbashir et al., 2001b).

Other studies have indicated that a 5′-phosphate on the target-complementary strand of a siRNA duplex is required for siRNA activity and that ATP is utilized to maintain the 5′-phosphate moiety on the siRNA (Nykanen et al., 2001). Other modifications that might be tolerated when introduced into an siRNA molecule include modifications of the sugar-phosphate backbone or the substitution of the nucleoside with at least one of a nitrogen or sulfur heteroatom (PCT International Publication Nos. WO 00/44914 and WO 01/68836) and certain nucleotide modifications that might inhibit the activation of double stranded RNA-dependent protein kinase (PKR), specifically 2′-amino or 2′-O-methyl nucleotides, and nucleotides containing a 2′-O or 4′-C methylene bridge (Canadian Patent Application No. 2,359,180).

Other references disclosing the use of dsRNA and RNAi include PCT International Publication Nos. WO 01/75164 (in vitro RNAi system using cells from Drosophila and the use of specific siRNA molecules for certain functional genomic and certain therapeutic applications); WO 01/36646 (methods for inhibiting the expression of particular genes in mammalian cells using dsRNA molecules); WO 99/32619 (methods for introducing dsRNA molecules into cells for use in inhibiting gene expression); WO 01/92513 (methods for mediating gene suppression by using factors that enhance RNAi); WO 02/44321 (synthetic siRNA constructs); WO 00/63364 and WO 01/04313 (methods and compositions for inhibiting the function of polynucleotide sequences); and WO 02/055692 and WO 02/055693 (methods for inhibiting gene expression using RNAi).

II. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims. Thus, for example, reference to “a vector” includes a plurality of such vectors, and so forth.

As used herein, the term “about,” when referring to a value or to an amount of mass, weight, time, volume, concentration or percentage is meant to encompass variations of ±20% or ±10%, in another example ±5%, in another example ±1%, and in still another example ±0.1% from the specified amount, as such variations are appropriate to perform the disclosed method.

As used herein, “significance” or “significant” relates to a statistical analysis of the probability that there is a non-random association between two or more entities. To determine whether or not a relationship is “significant” or has “significance”, statistical manipulations of the data can be performed to calculate a probability, expressed as a “p-value”. Those p-values that fall below a user-defined cutoff point are regarded as significant. In one example, a p-value less than or equal to 0.05, in another example less than 0.01, in another example less than 0.005, and in yet another example less than 0.001, are regarded as significant.

As used herein, the phrase “target RNA” refers to an RNA molecule (for example, an mRNA molecule encoding a hypoxia-inducible gene product) that is a target for downregulation. Similarly, the phrase “target site” refers to a sequence within a target RNA that is “targeted” for cleavage mediated by an siRNA construct that contains sequences within its antisense strand that are complementary to the target site. Also similarly, the phrase “target cell” refers to a cell that expresses a target RNA and into which an siRNA is intended to be introduced. A target cell is in some embodiments a cell in a subject. For example, a target cell can comprise a cell that expresses a hypoxia-inducible gene.

As used herein, the phrase “detectable level of cleavage” refers to a degree of cleavage of target RNA (and formation of cleaved product RNAS) that is sufficient to allow detection of cleavage products above the background of RNAs produced by random degradation of the target RNA. Production of siRNA-mediated cleavage products from at least 1-5% of the target RNA is sufficient to allow detection above background for most detection methods.

The terms “small interfering RNA”, “short interfering RNA”, and “siRNA” are used interchangeably and refer to any nucleic acid molecule capable of mediating RNA interference (RNAi) or gene silencing. See e.g., Bass, 2001; Elbashir et al., 2001a; and PCT International Publication Nos. WO 00/44895, WO 01/36646, WO 99/32619, WO 00/01846, WO 01/29058, WO 99/07409, and WO 00/44914. A non-limiting example of an siRNA molecule of the presently disclosed subject matter is shown in SEQ ID NO: 5. In some embodiments, the siRNA comprises a double stranded polynucleotide molecule comprising complementary sense and antisense regions, wherein the antisense region comprises a sequence complementary to a region of a target nucleic acid molecule (for example, an mRNA encoding HIF-1α). In some embodiments, the siRNA comprises a single stranded polynucleotide having self-complementary sense and antisense regions, wherein the antisense region comprises a sequence complementary to a region of a target nucleic acid molecule. In some embodiments, the siRNA comprises a single stranded polynucleotide having one or more loop structures and a stem comprising self complementary sense and antisense regions, wherein the antisense region comprises a sequence complementary to a region of a target nucleic acid molecule, and wherein the polynucleotide can be processed either in vivo or in vitro to generate an active siRNA capable of mediating RNAi. As used herein, siRNA molecules need not be limited to those molecules containing only RNA, but further encompass chemically modified nucleotides and non-nucleotides.

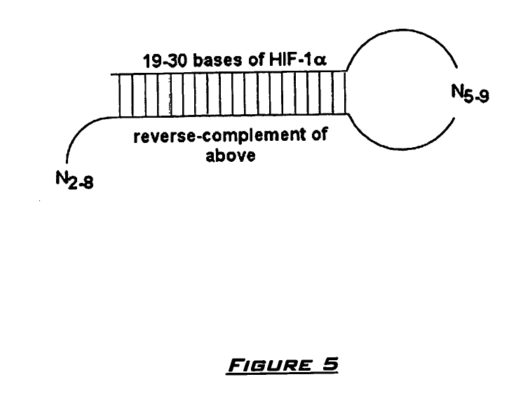

The siRNA molecules of the presently disclosed subject matter include, but are not limited to an siRNA molecule of the general structure depicted in FIG. 5. For the double-stranded molecule shown in FIG. 5, N can be any nucleotide, provided that in the loop structure identified as N₅₋₉, all 5-9 nucleotides remain in a single-stranded conformation. Similarly, N₂₋₈ can be any sequence of 2-8 nucleotides or modified nucleotides, provided that the nucleotides remain in a single-stranded conformation in the siRNA molecule. The duplex represented in FIG. 5 as “19-30 bases of HIF-1α” can be formed using any contiguous 19-30 base sequence of one of the HIF-1α gene products disclosed herein (for example, SEQ ID NOs: 1 and 3, but also including, for example, those HIF-1α gene products disclosed in GENBANK® Accession Nos: NM_{—}174339, NM_{—}024359, AJ439692, and AJ439691). In constructing an siRNA molecule of the presently disclosed subject matter, this 19-30 base sequence is followed (in a 5′ to 3′ direction) by 5-9 random nucleotides (N₅₋₉ above), the reverse-complement of the 19-30 base sequence, and finally 2-8 random nucleotides (N₂₋₈ above). This generic structure is exemplified by SEQ ID NO: 5. In some embodiments, an siRNA molecule of the presently disclosed subject matter comprises the nucleotide sequence presented in SEQ ID NO: 6.

The term “gene expression” generally refers to the cellular processes by which a biologically active polypeptide is produced from a DNA sequence and exhibits a biological activity in a cell. As such, gene expression involves the processes of transcription and translation, but also involves post-transcriptional and post-translational processes that can influence a biological activity of a gene or gene product. These processes include, but are not limited to RNA syntheses, processing, and transport, as well as polypeptide synthesis, transport, and post-translational modification of polypeptides. Additionally, processes that affect protein-protein interactions within the cell (for example, the interaction between HIF-1α and pVHL) can also affect gene expression as defined herein.

As used herein, the term “modulate” refers to a change in the expression level of a gene, or a level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator. For example, the term “modulate” can mean “inhibit” or “suppress”, but the use of the word “modulate” is not limited to this definition.

As used herein, the terms “inhibit”, “suppress”, “down regulate”, and grammatical variants thereof are used interchangeably and refer to an activity whereby gene expression or a level of an RNA encoding one or more gene products is reduced below that observed in the absence of a nucleic acid molecule of the presently disclosed subject matter. In some embodiments, inhibition with an siRNA molecule results in a decrease in the steady state level of a target RNA. In some embodiments, inhibition with a siRNA molecule results in an expression level of a target gene that is below that level observed in the presence of an inactive or attenuated molecule that is unable to mediate an RNAi response. In some embodiments, inhibition of gene expression with an siRNA molecule of the presently disclosed subject matter is greater in the presence of the siRNA molecule than in its absence. In some embodiments, inhibition of gene expression is associated with an enhanced rate of degradation of the mRNA encoded by the gene (for example, by RNAi mediated by an siRNA).

As used herein, the terms “gene” and “target gene” refer to a nucleic acid that encodes an RNA, for example, nucleic acid sequences including, but not limited to, structural genes encoding a polypeptide. The target gene can be a gene derived from a cell, an endogenous gene, a transgene, or exogenous genes such as genes of a pathogen, for example a virus, which is present in the cell after infection thereof. The cell containing the target gene can be derived from or contained in any organism, for example a plant, animal, protozoan, virus, bacterium, or fungus. The term “gene” also refers broadly to any segment of DNA associated with a biological function. As such, the term “gene” encompasses sequences including but not limited to a coding sequence, a promoter region, a transcriptional regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

In some embodiments, a gene is a hypoxia-inducible gene. As used herein, a “hypoxia-inducible gene” is a gene for which the expression level increases in response to hypoxia. In some embodiments, a hypoxia-inducible gene is a gene that is characterized by upregulated transcription in response to hypoxic conditions. Exemplary hypoxia-inducible genes thus include genes with hypoxia response elements (HREs) in their promoters. Under hypoxic conditions, transcription of these genes is induced as a result of activated HIF-1 binding to the HREs. Also as used herein, a hypoxia-inducible gene is a gene for which an activity of the gene product changes in response to hypoxia. In this embodiment, a hypoxia-inducible gene is a gene for which the polypeptide encoded by the gene experiences a change in state in response to hypoxia. Such a change in state includes, but is not limited to a post-transcriptional modification or an interaction with another molecule (for example, a protein-protein interaction). Thus, as used herein, the term hypoxia-inducible gene includes HIF-1α and pVHL, each of which undergoes a change in state (in this example, a dissociation one from the other) in response to hypoxia.

As is understood in the art, a gene comprises a coding strand and a non-coding strand. As used herein, the terms “coding strand” and “sense strand” are used interchangeably, and refer to a nucleic acid sequence that has the same sequence of nucleotides as an mRNA from which the gene product is translated. As is also understood in the art, when the coding strand and/or sense strand is used to refer to a DNA molecule, the coding/sense strand includes thymidine residues instead of the uridine residues found in the corresponding mRNA. Additionally, when used to refer to a DNA molecule, the coding/sense strand can also include additional elements not found in the mRNA including, but not limited to promoters, enhancers, and introns. Similarly, the terms “template strand” and “antisense strand” are used interchangeably and refer to a nucleic acid sequence that is complementary to the coding/sense strand.

As used herein, the terms “complementarity” and “complementary” refer to a nucleic acid that can form one or more hydrogen bonds with another nucleic acid sequence by either traditional Watson-Crick or other non-traditional types of interactions. In reference to the nucleic molecules of the presently disclosed subject matter, the binding free energy for a nucleic acid molecule with its complementary sequence is sufficient to allow the relevant function of the nucleic acid to proceed, in some embodiments, RNAi activity. For example, the degree of complementarity between the sense and antisense strands of the siRNA construct can be the same or different from the degree of complementarity between the antisense strand of the siRNA and the target nucleic acid sequence. Complementarity to the target sequence of less than 100% in the antisense strand of the siRNA duplex, including point mutations, is not well tolerated when these changes are located between the 3′-end and the middle of the antisense siRNA, whereas mutations near the 5′-end of the antisense siRNA strand can exhibit a small degree of RNAi activity (Elbashir et al., 2001c). Determination of binding free energies for nucleic acid molecules is well known in the art. See e.g., Freier et al., 1986; Tumer et al., 1987.

As used herein, the phrase “percent complementarity” refers to the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). The terms “100% complementary”, “fully complementary”, and “perfectly complementary” indicate that all of the contiguous residues of a nucleic acid sequence can hydrogen bond with the same number of contiguous residues in a second nucleic acid sequence.

As used herein, the term “cell” is used in its usual biological sense. In some embodiments, the cell is present in an organism, for example, mammals such as humans, cows, sheep, apes, monkeys, swine, dogs, and cats. The cell can be eukaryotic (e.g., a mammalian cell, such as a human cell) or prokaryotic (e.g. a bacterium). The cell can be of somatic or germ line origin, totipotent or pluripotent, dividing or non-dividing. The cell can also be derived from or can comprise a gamete or embryo, a stem cell, or a fully differentiated cell.

The siRNA molecules of the presently disclosed subject matter can be added directly to the cell, or can be complexed with cationic lipids, packaged within liposomes, or otherwise delivered to target cells or tissues. The nucleic acid or nucleic acid complexes can be locally administered to relevant tissues ex vivo, or in vivo through injection, infusion pump or stent, with or without their incorporation into biopolymers. In a particular embodiment, a nucleic acid molecule of the presently disclosed subject matter comprises the sequence shown in SEQ ID NO: 6. Alternatively, the siRNA molecule of the presently disclosed subject matter can be encoded by a recombinant vector (for example, a viral vector).

As used herein, the term “RNA” refers to a molecule comprising at least one ribonucleotide residue. By “ribonucleotide” is meant a nucleotide with a hydroxyl group at the 2′ position of a β-D-ribofuranose moiety. The terms encompass double stranded RNA, single stranded RNA, RNAs with both double stranded and single stranded regions, isolated RNA such as partially purified RNA, essentially pure RNA, synthetic RNA, recombinantly produced RNA, as well as altered RNA, or analog RNA, that differs from naturally occurring RNA by the addition, deletion, substitution, and/or alteration of one or more nucleotides. Such alterations can include addition of non-nucleotide material, such as to the end(s) of the siRNA or internally, for example at one or more nucleotides of the RNA. Nucleotides in the RNA molecules of the presently disclosed subject matter can also comprise non-standard nucleotides, such as non-naturally occurring nucleotides or chemically synthesized nucleotides or deoxynucleotides. These altered RNAs can be referred to as analogs or analogs of a naturally occurring RNA.

As used herein, the phrase “double stranded RNA” refers to an RNA molecule at least a part of which is in Watson-Crick base pairing forming a duplex. As such, the term is to be understood to encompass an RNA molecule that is either fully or only partially double stranded. Exemplary double stranded RNAs include, but are not limited to molecules comprising at least two distinct RNA strands that are either partially or fully duplexed by intermolecular hybridization. Additionally, the term is intended to include a single RNA molecule that by intramolecular hybridization can form a double stranded region (for example, a hairpin). Thus, as used herein the phrases “intermolecular hybridization” and “intramolecular hybridization” refer to double stranded molecules for which the nucleotides involved in the duplex formation are present on different molecules or the same molecule, respectively.

As used herein, the phrase “double stranded region” refers to any region of a nucleic acid molecule that is in a double stranded conformation via hydrogen bonding between the nucleotides including, but not limited to hydrogen bonding between cytosine and guanosine, adenosine and thymidine, adenosine and uracil, and any other nucleic acid duplex as would be understood by one of ordinary skill in the art. The length of the double stranded region can vary from about 15 consecutive basepairs to several thousand basepairs. In some embodiments, the double stranded region is at least 15 basepairs, in some embodiments between 15 and 50 basepairs, and in some embodiments between 15 and 30 basepairs. In some embodiments, the length of the double stranded region is selected from the group consisting of 19, 20, 21, 22, 23, 24, 25, 26, 27, 28, 29, and 30 basepairs. In some embodiments, the double stranded region comprises a first strand comprising a ribonucleotide sequence that corresponds to a coding strand of the hypoxia-inducible gene and a second strand comprising a ribonucleotide sequence that is complementary to the first strand, and wherein the first strand and the second strand hybridize to each other to form the double-stranded molecule. As used herein, the terms “corresponds to”, “corresponding to”, and grammatical variants thereof refer to a nucleotide sequence that is 100% identical to at least 19 contiguous nucleotides of a nucleic acid sequence of a hypoxia-inducible gene. Thus, a first nucleic acid sequence that “corresponds to” a coding strand of a hypoxia-inducible gene is a nucleic acid sequence that is 100% identical to at least 19 contiguous nucleotides of a hypoxia-inducible gene, including, but note limited to 5′ untranslated sequences, exon sequences, intron sequences, and 3′ untranslated sequences.

In a representative embodiment, the length of the double stranded region is 19 basepairs. As describe hereinabove, the formation of the double stranded region results from the hybridization of complementary RNA strands (for example, a sense strand and an antisense strand), either via an intermolecular hybridization (i.e. involving 2 or more distinct RNA molecules) or via an intramolecular hybridization, the latter of which can occur when a single RNA molecule contains self-complementary regions that are capable of hybridizing to each other on the same RNA molecule. These self-complementary regions are typically separated by a short stretch of nucleotides (for example, about 5-10 nucleotides) such that the intramolecular hybridization event forms what is referred to in the art as a “hairpin”.

The nucleic acid molecules of the presently disclosed subject matter individually, or in combination or in conjunction with other drugs, can be used to treat diseases or conditions discussed herein. For example, to treat a particular disease or condition, the siRNA molecules can be administered to a subject or can be administered to other appropriate cells evident to those skilled in the art, individually or in combination with one or more drugs under conditions suitable for the treatment.

III. Nucleic Acids

The nucleic acid molecules employed in accordance with the presently disclosed subject matter include any nucleic acid molecule encoding a hypoxia-inducible gene product, as well as the nucleic acid molecules that are used in accordance with the presently disclosed subject matter to a modulation of the expression of the hypoxia-inducible gene. Thus, the nucleic acid molecules employed in accordance with the presently disclosed subject matter include, but are not limited to, the nucleic acid molecules shown in SEQ ID NOs: 1, 3, 5, and 6; sequences substantially identical to SEQ ID NOs: 1, 3, 5, and 6; and subsequences and elongated sequences thereof. The presently disclosed subject matter also encompasses genes, cDNAs, chimeric genes, and vectors comprising disclosed nucleic acid sequences.

The term “nucleic acid molecule” refers to deoxyribonucleotides or ribonucleotides and polymers thereof in either single- or double-stranded form. Unless specifically limited, the term encompasses nucleic acids containing known analogues of natural nucleotides that have similar properties as the reference natural nucleic acid. Unless otherwise indicated, a particular nucleotide sequence also implicitly encompasses complementary sequences, subsequences, elongated sequences, as well as the sequence explicitly indicated. The terms “nucleic acid molecule” or “nucleotide sequence” can also be used in place of “gene”, “DNA”, “cDNA”, “RNA”, or “mRNA”. Nucleic acids can be derived from any source, including any organism.

The term “isolated”, as used in the context of a nucleic acid molecule, indicates that the nucleic acid molecule exists apart from its native environment and is not a product of nature. An isolated DNA molecule can exist in a purified form or can exist in a non-native environment such as a transgenic host cell.

The terms “identical” or percent “identity” in the context of two or more nucleotide or polypeptide sequences, refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

The term “substantially identical”, in the context of two nucleotide sequences, refers to two or more sequences or subsequences that in one example have at least 60%, in another example about 70%, in another example about 80%, in another example about 90-95%, and in yet another example about 99% nucleotide identity, when compared and aligned for maximum correspondence, as measured using one of the following sequence comparison algorithms (described herein below) or by visual inspection. In one example, the substantial identity exists in nucleotide sequences of at least 50 residues, in another example in nucleotide sequence of at least about 100 residues, in another example in nucleotide sequences of at least about 150 residues, and in yet another example in nucleotide sequences comprising complete coding sequences.

In one aspect, polymorphic sequences can be substantially identical sequences. The terms “polymorphic”, “polymorphism”, and “polymorphic variants” refer to the occurrence of two or more genetically determined alternative sequences or alleles in a population. An allelic difference can be as small as one base pair. As used herein in regards to a nucleotide or polypeptide sequence, the term “substantially identical” also refers to a particular sequence that varies from another sequence by one or more deletions, substitutions, or additions, the net effect of which is to retain biological activity of a gene, gene product, or sequence of interest.

For sequence comparison, typically one sequence acts as a reference sequence to which test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison can be conducted, e.g., by the local homology algorithm of Smith & Waterman, 1981, by the homology alignment algorithm of Needleman & Wunsch, 1970, by the search for similarity method of Pearson & Lipman, 1988, by computerized implementations of these algorithms (GAP, BESTFIT, FASTA, and TFASTA, in the Wisconsin Genetics Software Package, available from Accelrys Inc., San Diego, Calif., United States of America), or by visual inspection. See generally, Ausubel, 1995.

In some embodiments, an algorithm for determining percent sequence identity and sequence similarity is the BLAST algorithm, which is described by Altschul et al., 1990. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff, 1992.

In addition to calculating percent sequence identity, the BLAST algorithm also performs a statistical analysis of the similarity between two sequences. See e.g., Karin & Altschul, 1993. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in some embodiments less than about 0.1, in some embodiments less than about 0.01, and in some embodiments less than about 0.001.

Another indication that two nucleotide sequences are substantially identical is that the two molecules specifically or substantially hybridize to each other under stringent conditions. In the context of nucleic acid hybridization, two nucleic acid sequences being compared can be designated a “probe” and a “target”. A “probe” is a reference nucleic acid molecule, and a “target” is a test nucleic acid molecule, often found within a heterogeneous population of nucleic acid molecules. A “target sequence” is synonymous with a “test sequence”.

An exemplary nucleotide sequence employed in the methods disclosed herein comprises sequences that are complementary to each other, the complementary regions being capable of forming a duplex of in some embodiments at least about 15 to 50 basepairs. One strand of the duplex comprises a nucleic acid sequence of at least 15 contiguous bases having a nucleic acid sequence of a nucleic acid molecule of the presently disclosed subject matter (for example, SEQ ID NOs: 1 or 3). In one example, one strand of the duplex comprises a nucleic acid sequence comprising 15 to 18 nucleotides, or even longer where desired, such as 19, 20, 21, 22, 25, or 30 nucleotides or up to the full length of any of those set forth as SEQ ID NOs: 1 and 3. Such fragments can be readily prepared by, for example, directly synthesizing the fragment by chemical synthesis, by application of nucleic acid amplification technology, or by introducing selected sequences into recombinant vectors for recombinant production. The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA).

The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization.

“Stringent hybridization conditions” and “stringent hybridization wash conditions” in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen, 1993. Generally, highly stringent hybridization and wash conditions are selected to be about 5° C. lower than the thermal melting point (Tₘ) for the specific sequence at a defined ionic strength and pH. Typically, under “stringent conditions” a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of highly stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1× standard saline citrate (SSC), 0.1% (w/v) SDS at 65° C. Another example of highly stringent wash conditions is 15 minutes in 0.2×SSC buffer at 65° C. (see Sambrook and Russell, 2001 for a description of SSC buffer and other stringency conditions). Often, a high stringency wash is preceded by a lower stringency wash to remove background probe signal. An example of medium stringency wash conditions for a duplex of more than about 100 nucleotides is 15 minutes in 1×SSC at 45° C. Another example of medium stringency wash for a duplex of more than about 100 nucleotides is 15 minutes in 4-6×SSC at 40° C. For short probes (e.g., about 10 to 50 nucleotides), stringent conditions typically involve salt concentrations of less than about 1M Na⁺ ion, typically about 0.01 to 1M Na⁺ ion concentration (or other salts) at pH 7.0-8.3, and the temperature is typically at least about 30° C. Stringent conditions can also be achieved with the addition of destabilizing agents such as formamide. In general, a signal to noise ratio of 2-fold or higher than that observed for an unrelated probe in the particular hybridization assay indicates detection of a specific hybridization.

The following are examples of hybridization and wash conditions that can be used to clone homologous nucleotide sequences that are substantially identical to reference nucleotide sequences of the presently disclosed subject matter: a probe nucleotide sequence hybridizes in one example to a target nucleotide sequence in 7% sodium dodecyl sulfate (SDS), 0.5M NaPO₄, 1 mm EDTA at 50° C. followed by washing in 2×SSC, 0.1% SDS at 50° C.; in another example, a probe and target sequence hybridize in 7% sodium dodecyl sulfate (SDS), 0.5M NaPO₄, 1 mm EDTA at 50° C. followed by washing in 1×SSC, 0.1% SDS at 50° C.; in another example, a probe and target sequence hybridize in 7% sodium dodecyl sulfate (SDS), 0.5M NaPO₄, 1 mm EDTA at 50° C. followed by washing in 0.5×SSC, 0.1% SDS at 50° C.; in another example, a probe and target sequence hybridize in 7% sodium dodecyl sulfate (SDS), 0.5M NaPO₄, 1 mm EDTA at 50° C. followed by washing in 0.1× SSC, 0.1% SDS at 50° C.; in yet another example, a probe and target sequence hybridize in 7% sodium dodecyl sulfate (SDS), 0.5M NaPO₄, 1 mm EDTA at 50° C. followed by washing in 0.1×SSC, 0.1% SDS at 65° C.

The term “subsequence” refers to a sequence of nucleic acids that comprises a part of a longer nucleic acid sequence. An exemplary subsequence is a sequence that comprises part of a duplexed region of an siRNA, one strand of which is complementary to the sequence of an mRNA.

The term “elongated sequence” refers to an addition of nucleotides (or other analogous molecules) incorporated into the nucleic acid. For example, a polymerase (e.g., a DNA polymerase) can add sequences at the 3′ terminus of the nucleic acid molecule. In addition, the nucleotide sequence can be combined with other DNA sequences, such as promoters, promoter regions, enhancers, polyadenylation signals, intronic sequences, additional restriction enzyme sites, multiple cloning sites, and other coding segments.

The terms “operatively linked” and “operably linked”, as used herein, refer to a promoter region that is connected to a nucleotide sequence in such a way that the transcription of that nucleotide sequence is controlled and regulated by that promoter region. Similarly, a nucleotide sequence is said to be under the “transcriptional control” of a promoter to which it is operably linked. Techniques for operatively linking a promoter region to a nucleotide sequence are known in the art.

The terms “heterologous gene”, “heterologous DNA sequence”, “heterologous nucleotide sequence”, “exogenous nucleic acid molecule”, or “exogenous DNA segment”, as used herein, each refer to a sequence that originates from a source foreign to an intended host cell or, if from the same source, is modified from its original form. Thus, a heterologous gene in a host cell includes a gene that is endogenous to the particular host cell but has been modified, for example by mutagenesis or by isolation from native transcriptional regulatory sequences. The terms also include non-naturally occurring multiple copies of a naturally occurring nucleotide sequence. Thus, the terms refer to a DNA segment that is foreign or heterologous to the cell, or homologous to the cell but in a position within the host cell nucleic acid wherein the element is not ordinarily found.

The term “expression vector” as used herein refers to a DNA sequence capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The construct comprising the nucleotide sequence of interest can be chimeric. The construct can also be one that is naturally occurring but has been obtained in a recombinant form useful for heterologous expression.

The term “promoter” or “promoter region” each refers to a nucleotide sequence within a gene that is positioned 5′ to a coding sequence of a same gene and functions to direct transcription of the coding sequence. The promoter region comprises a transcriptional start site, and can additionally include one or more transcriptional regulatory elements. In some embodiments, a method of the presently disclosed subject matter employs a hypoxia inducible promoter.

A “minimal promoter” is a nucleotide sequence that has the minimal elements required to enable basal level transcription to occur. As such, minimal promoters are not complete promoters but rather are subsequences of promoters that are capable of directing a basal level of transcription of a reporter construct in an experimental system. Minimal promoters include but are not limited to the CMV minimal promoter, the HSV-tk minimal promoter, the simian virus 40 (SV40) minimal promoter, the human β-actin minimal promoter, the human EF2 minimal promoter, the adenovirus E1B minimal promoter, and the heat shock protein (hsp) 70 minimal promoter. Minimal promoters are often augmented with one or more transcriptional regulatory elements to influence the transcription of an operably linked gene. For example, cell-type-specific or tissue-specific transcriptional regulatory elements can be added to minimal promoters to create recombinant promoters that direct transcription of an operably linked nucleotide sequence in a cell-type-specific or tissue-specific manner

Different promoters have different combinations of transcriptional regulatory elements. Whether or not a gene is expressed in a cell is dependent on a combination of the particular transcriptional regulatory elements that make up the gene's promoter and the different transcription factors that are present within the nucleus of the cell. As such, promoters are often classified as “constitutive”, “tissue-specific”, “cell-type-specific”, or “inducible”, depending on their functional activities in vivo or in vitro. For example, a constitutive promoter is one that is capable of directing transcription of a gene in a variety of cell types. Exemplary constitutive promoters include the promoters for the following genes which encode certain constitutive or “housekeeping” functions: hypoxanthine phosphoribosyl transferase (HPRT), dihydrofolate reductase (DHFR; (Scharfmann et al., 1991), adenosine deaminase, phosphoglycerate kinase (PGK), pyruvate kinase, phosphoglycerate mutase, the β-actin promoter (see, e.g. Williams et al., 1993), and other constitutive promoters known to those of skill in the art. “Tissue-specific” or “cell-type-specific” promoters, on the other hand, direct transcription in some tissues and cell types but are inactive in others. Exemplary tissue-specific promoters include the PSA promoter (Yu et al., 1999; Lee et al., 2000), the probasin promoter (Greenberg et al., 1994; Yu et al., 1999), and the MUC1 promoter (Kurihara et al., 2000) as discussed above, as well as other tissue-specific and cell-type specific promoters known to those of skill in the art.

When used in the context of a promoter, the term “linked” as used herein refers to a physical proximity of promoter elements such that they function together to direct transcription of an operably linked nucleotide sequence

The term “transcriptional regulatory sequence” or “transcriptional regulatory element”, as used herein, each refers to a nucleotide sequence within the promoter region that enables responsiveness to a regulatory transcription factor. Responsiveness can encompass a decrease or an increase in transcriptional output and is mediated by binding of the transcription factor to the DNA molecule comprising the transcriptional regulatory element.

The term “transcription factor” generally refers to a protein that modulates gene expression by interaction with the transcriptional regulatory element and cellular components for transcription, including RNA Polymerase, Transcription Associated Factors (TAFs), chromatin-remodeling proteins, and any other relevant protein that impacts gene transcription.

The terms “reporter gene” or “marker gene” or “selectable marker” each refer to a heterologous gene encoding a product that is readily observed and/or quantitated. A reporter gene is heterologous in that it originates from a source foreign to an intended host cell or, if from the same source, is modified from its original form. Non-limiting examples of detectable reporter genes that can be operatively linked to a transcriptional regulatory region can be found in Alam & Cook, 1990 and PCT International Publication No. WO 97/47763. Exemplary reporter genes for transcriptional analyses include the lacZ gene (see e.g., Rose & Botstein, 1983), Green Fluorescent Protein (GFP; Cubitt et al, 1995), luciferase, and chloramphenicol acetyl transferase (CAT). Reporter genes for methods to produce transgenic animals include but are not limited to antibiotic resistance genes, for example the antibiotic resistance gene confers neomycin resistance. Any suitable reporter and detection method can be used, and it will be appreciated by one of skill in the art that no particular choice is essential to or a limitation of the presently disclosed subject matter.

An amount of reporter gene can be assayed by any method for qualitatively or quantitatively determining presence or activity of the reporter gene product. The amount of reporter gene expression directed by each test promoter region fragment is compared to an amount of reporter gene expression to a control construct comprising the reporter gene in the absence of a promoter region fragment. A promoter region fragment is identified as having promoter activity when there is significant increase in an amount of reporter gene expression in a test construct as compared to a control construct. The term “significant increase”, as used herein, refers to an quantified change in a measurable quality that is larger than the margin of error inherent in the measurement technique, in one example an increase by about 2-fold or greater relative to a control measurement, in another example an increase by about 5-fold or greater, and in yet another example an increase by about 10-fold or greater.

The presently disclosed subject matter includes in some embodiments adenovirus vectors comprising the disclosed nucleotide sequences. The term “vector”, as used herein refers to a DNA molecule having sequences that enable the transfer of those sequences to a compatible host cell. A vector also includes nucleotide sequences to permit ligation of nucleotide sequences within the vector, wherein such nucleotide sequences are also replicated in a compatible host cell. A vector can also mediate recombinant production of a therapeutic polypeptide, as described further herein below.

Nucleic acids of the presently disclosed subject matter can be cloned, synthesized, recombinantly altered, mutagenized, or combinations thereof. Standard recombinant DNA and molecular cloning techniques used to isolate nucleic acids are known in the art. Exemplary, non-limiting methods are described by Silhavy et al., 1984; Ausubel et al., 1992; Glover & Hames, 1995; and Sambrook & Russell, 2001). Site-specific mutagenesis to create base pair changes, deletions, or small insertions is also known in the art as exemplified by publications (see e.g., Adelman et al., 1983; Sambrook & Russell, 2001).

III.A. Synthesis of Nucleic Acid Molecules

In one aspect, the presently disclosed subject matter provides an siRNA molecule that has been synthesized outside of a target cell prior to introduction of the siRNA into the target cell. In this embodiment, the synthesis can be performed either mechanically (i.e., using an RNA synthesis machine) or using recombinant techniques.

Mechanical synthesis of nucleic acids greater than 100 nucleotides in length is difficult using automated methods, and the cost of such molecules tends to be prohibitive. As used herein, small nucleic acid motifs (“small” referring to nucleic acid motifs in some embodiments no more than 100 nucleotides in length, in some embodiments no more than 80 nucleotides in length, and in some embodiments no more than 50 nucleotides in length; e.g., individual siRNA oligonucleotide sequences or siRNA sequences synthesized in tandem) can be used for exogenous delivery. The simple structure of these molecules increases the ability of the nucleic acid to invade targeted regions of protein and/or RNA structure. Exemplary molecules of the presently disclosed subject matter are chemically synthesized, and others can similarly be synthesized.

Oligonucleotides (e.g., certain modified oligonucleotides or portions of oligonucleotides lacking ribonucleotides) are synthesized using protocols known in the art. See e.g., Caruthers et al., 1992; PCT International Publication No. WO 99/54459; Wincott et al., 1995; Wincott & Usman, 1997; Brennan et al., 1998; and U.S. Pat. No. 6,001,311, each of which is incorporated herein by reference. The synthesis of oligonucleotides makes lse of common nucleic acid protecting and coupling groups, such as dimethoxytrityl at the 5′-end, and phosphoramidites at the 3′-end. In a non-limiting example, small-scale syntheses can be conducted on a Applied Biosystems 3400 DNA Synthesizer (Applied Biosystems Inc., Foster City, Calif., United States of America) using a 0.2 μmol scale protocol with a 2.5 minute coupling step for 2′-O-methylated nucleotides and a 45 second coupling step for 2-deoxy nucleotides or 2′-deoxy-2′-fluoro nucleotides. Alternatively, syntheses at the 0.2 μmol scale can be performed on a 96-well plate synthesizer. A 33-fold excess (60 μL of 0.11 M; 6.6 μmol) of 2′-O-methyl phosphoramidite and a 105-fold excess of S-ethyl tetrazole (60 μL of 0.25 M; 15 μmol) can be used in each coupling cycle of 2′-O-methyl residues relative to polymer-bound 5′-hydroxyl. A 22-fold excess (40 μL of 0.11 M; 4.4 μmol) of deoxy phosphoramidite and a 70-fold excess of S-ethyl tetrazole (40 μL of 0.25 M; 10 μmol) can be used in each coupling cycle of deoxy residues relative to polymer-bound 5′-hydroxyl. Average coupling yields on the Applied Biosystems 3400 DNA Synthesizer, determined by colorimetric quantitation of the trityl fractions, are typically 97.5-99%. Other oligonucleotide synthesis reagents for the Applied Biosystems 3400 DNA Synthesizer include the following: detritylation solution is 3% TCA in methylene chloride (Applied Biosystems, Inc.); capping is performed with 16% N-methyl imidazole in THF (Applied Biosystems, Inc.) and 10% acetic anhydride/10% 2,6-lutidine in THF (Applied Biosystems, Inc.); and oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate internucleotide linkages, Beaucage reagent (³H-1,2-benzodithiol-3-one 1,1-dioxide, 0.05 M in acetonitrile) is used.

Deprotection of the DNA-based oligonucleotides is performed as follows: the polymer-bound trityl-on oligoribonucleotide is transferred to a 4 mL glass screw top vial and suspended in a solution of 40% aqueous methylamine (1 mL) at 65° C. for 10 minutes. After cooling to −20° C., the supernatant is removed from the polymer support. The support is washed three times with 1.0 mL of EtOH:MeCN:H₂O (3:1:1), vortexed, and the supernatant is added to the first supernatant. The combined supernatants, containing the oligoribonucleotide, are dried to a white powder.

In some embodiments, the method of synthesis used for RNA including certain siRNA molecules of the presently disclosed subject matter follows the procedure as described in Usman et al., 1987; Scaringe et al., 1990; Wincott et al., 1995; Wincott & Usman, 1997; and makes use of common nucleic acid protecting and coupling groups, such as dimethoxytrityl at the 5′-end, and phosphoramidites at the 3′-end. In a non-limiting example, small-scale syntheses are conducted on an Applied Biosystems 3400 DNA Synthesizer using a 0.2 μmol scale protocol with a 7.5 minute coupling step for alkylsilyl protected nucleotides and a 2.5 minute coupling step for 2′-O-methylated nucleotides. Alternatively, syntheses at the 0.2 μmol scale can be done on a 96-well plate synthesizer. A 33-fold excess (60 μL of 0.11 M; 6.6 μmol) of 2′-O-methyl phosphoramidite and a 75-fold excess of S-ethyl tetrazole (60 μL of 0.25 M; 15.5 mol) can be used in each coupling cycle of 2′-O-methyl residues relative to polymer-bound 5′-hydroxyl. A 66-fold excess (120 μL of 0.11 M; 13.2 μmol) of alkylsilyl (ribo) protected phosphoramidite and a 150-fold excess of S-ethyl tetrazole (120 μL of 0.25 M; 30 μmol) can be used in each coupling cycle of ribo residues relative to polymer-bound 5′-hydroxyl. Average coupling yields on the Applied Biosystems 3400 DNA Synthesizer, determined by calorimetric quantitation of the trityl fractions, are typically 97.5-99%. Other oligonucleotide synthesis reagents for the Applied Biosystems 3400 DNA Synthesizer include the following: detritylation solution is 3% TCA in methylene chloride (Applied Biosystems, Inc.); capping is performed with 16% N-methyl imidazole in THF (Applied Biosystems, Inc.) and 10% acetic anhydride/10% 2,6-lutidine in THF (Applied Biosystems, Inc.); oxidation solution is 16.9 mM 12, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. (Natick, Mass., United States of America). Alternately, for the introduction of phosphorothioate linkages, Beaucage reagent (3H-1,2-Benzodithiol-3-one 1,1-dioxide0.05 M in acetonitrile) is used.

Deprotection of the RNA can be performed, for example, using either a two-pot or one-pot protocol. For the two-pot protocol, the polymer-bound trityl-on oligoribonucleotide is transferred to a 4 mL glass screw top vial and suspended in a solution of 40% aqueous methylamine (1 mL) at 65° C. for 10 minutes. After cooling to −20° C., the supernatant is removed from the polymer support. The support is washed three times with 1.0 mL of EtOH:MeCN:H2O (3:1:1), vortexed, and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA-3HF to provide a 1.4 M HF concentration) and heated to 65° C. After 1.5 hours, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine:DMSO (1:1; 0.8 mL) at 65° C. for 15 minutes. The vial is brought to room temperature, TEA.3HF (0.1 mL) is added, and the vial is heated at 65° C. for 15 minutes. The sample is cooled at −20° C., and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEM. After washing the loaded cartridge with water, the RNA is detritylated with 0.5% trifluoroacetic acid (TFA) for 13 min. The cartridge is then washed again with water, salt exchanged with 1 M NaCl, and washed with water again. The oligonucleotide is then eluted with 30% acetonitrile.

The average stepwise coupling yields are typically greater than 98% (Wincott et al., 1995). Those of ordinary skill in the art will recognize that the scale of synthesis can be adapted to be larger or smaller than the example described above including but not limited to 96-well format: all that is important is the ratio of chemicals used in the reaction.

Alternatively, the nucleic acid molecules of the presently disclosed subject matter can be synthesized separately and joined together post-synthetically, for example, by ligation (PCT International Publication No. WO 93/23569; Shabarova et al., 1991; Bellon et al., 1997), or by hybridization following synthesis and/or deprotection.

The siRNA molecules of the presently disclosed subject matter can also be synthesized via a tandem synthesis methodology as described in Example 2 herein, wherein both siRNA strands are synthesized as a single contiguous oligonucleotide fragment or a strand separated by a linker which, in some embodiments, is subsequently cleaved to provide separate siRNA fragments or strands that hybridize and permit purification of the siRNA duplex. The linker can be a polynucleotide linker or a non-nucleotide linker. The tandem synthesis of siRNA as described herein can be readily adapted to both multiwell and multiplate synthesis platforms such as 96 well or similarly larger multi-well platforms. The tandem synthesis of siRNA as described herein can also be readily adapted to large-scale synthesis platforms employing batch reactors, synthesis columns and the like.

A siRNA molecule can also be assembled from two distinct nucleic acid strands or fragments wherein one fragment includes the sense region and the second fragment includes the antisense region of the RNA molecule.

The nucleic acid molecules of the presently disclosed subject matter can be modified extensively to enhance stability by modification with nuclease resistant groups including, but not limited to 2′-amino, 2′-C-allyl, 2′-fluoro, 2′-O-methyl, 2′-H (for a review see Usman & Cedergren, 1992; Usman et al., 1994). siRNA constructs can be purified by gel electrophoresis using general methods or can be purified by high pressure liquid chromatography (HPLC; see Wincott et al., 1995, the totality of which is hereby incorporated herein by reference) and re-suspended in water.

In some embodiments, recombinant techniques can be used to synthesize an siRNA, which can thereafter be purified from the source and transferred to a target cell. There are many techniques that are known in the art for the synthesis RNA molecules in recombinant cells, and any such technique can be used in the practice of the presently disclosed subject matter. One such general strategy for synthesizing an RNA molecule includes cloning a DNA sequence downstream of an RNA polymerase promoter and introducing the recombinant molecule into a cell in which the promoter is competent to direct transcription of the cloned sequence. This can be accomplished using a plasmid constructed for this purpose.

Alternatively, the RNA can be synthesized in the target cell using an expression vector, for example an expression plasmid. Such plasmids include, but are not limited to the pSILENCER™ series of plasmids (Ambion, Inc., Austin, Tex., United States of America), and the plasmid disclosed by Miyagishi & Taira, 2002.

The pSILENCER™ series of plasmids contain a cloning site downstream of a mammalian RNA polymerase III promoter. A nucleic acid encoding a hairpin with a 19 base pair duplex region can be cloned into the cloning site of one of these plasmids. When the recombinant plasmid is introduced into a mammalian cell, the RNA polymerase III promoter directs transcription of the hairpin RNA molecule, which thereafter forms the hairpin characterized by the 19 base pair duplex. This hairpin is apparently recognized by the Dicer nuclease, which cleaves the hairpin to form a functional siRNA.

Miyagishi & Taira, 2002, disclose another strategy for producing siRNA molecules. This reference discloses a plasmid that has two RNA polymerase III promoters. To produce an siRNA, the same 19 base pair nucleic acid molecule is cloned downstream of each promoter, but in opposite orientations. Thus, the plasmid produces distinct sense and antisense RNA strands, which then undergo intermolecular hybridization to produce an siRNA. In this case, the promoter is the U6 promoter. An RNA transcribed from a U6 promoter has a stretch of about four uridines at its 3′ end. Thus, the use of this plasmid results in the production of two RNA strands, each of which contains a 19 base region that is capable of hybridizing to a 19 base region in the other, with a short 3′ overhang.

III.B. Optimizing Activity of Nucleic Acid Molecules

Chemically synthesizing nucleic acid molecules incorporating various modifications (e.g. to base, sugar, and/or phosphate moieties) can reduce the degradation of the nucleic acid molecules by ribonucleases present in biological fluids, and can thus can increase the potency of therapeutic nucleic acid molecules (see e.g., PCT International Publication Nos. WO 92/07065, WO 93/15187, and WO 91/03162; U.S. Pat. Nos. 5,334,711 and 6,300,074; Perrault et al., 1990; Pieken et al., 1991; Usman & Cedergren, 1992; and Burgin et al., 1996; all of which are incorporated by reference herein). Each of the above references describe various chemical modifications that can be made to the base, phosphate, and/or sugar moieties of the nucleic acid molecules described herein. Modifications can be employed to enhance the efficacy of the disclosed nucleic acid molecules in cells.

There are several examples in the art describing sugar, base, and phosphate modifications that can be introduced into nucleic acid molecules with significant enhancement in their nuclease stability and efficacy. For example, oligonucleotides can be modified to enhance their stability and/or enhance biological activity by modification with nuclease resistant groups, for example, 2′-amino, 2′-C-allyl, 2′-fluoro, 2′-O-methyl, 2′-O-allyl, 2′-H, nucleotide base modifications (reviewed in Usman & Cedergren, 1992; Usman et al., 1994; Burgin et al., 1996). Sugar modification of nucleic acid molecules have been extensively described in the art (see PCT International Publication Nos. WO 92/07065, WO 93/15187, WO 98/13526, and WO 97/26270; U.S. Pat. Nos. 5,334,711; 5,716,824; and 5,627,053; Perrault et al., 1990; Pieken et al., 1991; Usman & Cedergren, 1992; Beigelman et al., 1995; Karpeisky et al., 1998; Earnshaw & Gait, 1998; Verma & Eckstein, 1998; Burlina et al., 1997; all of which are incorporated by reference herein). Such publications describe general methods and strategies to determine the location of incorporation of sugar, base, and/or phosphate modifications and the like into nucleic acid molecules without modulating catalysis. In view of such teachings, similar modifications can be used as described herein to modify the siRNA nucleic acid molecules of the presently disclosed subject matter so long as the ability of the siRNAs to promote RNAi in a cell is not significantly inhibited.

While chemical modification of oligonucleotide internucleotide linkages with phosphorothioate and/or 5′-methylphosphonate linkages improves stability, excessive modifications can cause toxicity or decreased activity. Therefore, when designing nucleic acid molecules, the number of these internucleotide linkages should be minimized. Reducing the concentration of these linkages should lower toxicity, resulting in increased efficacy and higher specificity of these molecules.

The term “universal base” as used herein refers to nucleotide base analogs that form base pairs with each of the natural DNA/RNA bases with little discrimination between them. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art (see, for example, Loakes, 2001).

Small interfering RNA (siRNA) molecules having chemical modifications that maintain or enhance activity are provided. Such a nucleic acid is also generally more resistant to nucleases than an unmodified nucleic acid. Accordingly, the in vitro and/or in vivo activity should not be significantly lowered. In cases in which modulation is the goal, nucleic acid molecules delivered exogenously should optimally be stable within cells until translation of the target RNA has been modulated long enough to reduce the levels of the undesirable protein. This period of time varies between hours to days depending upon the disease state. Improvements in the chemical synthesis of RNA (Wincott et al., 1995; Caruthers et al., 1992) have expanded the ability to modify nucleic acid molecules by introducing nucleotide modifications to enhance their nuclease stability, as described above.

In some embodiments, the presently disclosed subject matter features conjugates and/or complexes of siRNA molecules. Such conjugates and/or complexes can be used to facilitate delivery of siRNA molecules into a biological system, such as a cell. The conjugates and complexes provided by the presently disclosed subject matter can impart therapeutic activity by transferring therapeutic compounds across cellular membranes, altering the pharmacokinetics of, and/or modulating the localization of nucleic acid molecules of the presently disclosed subject matter. The presently disclosed subject matter encompasses the design and synthesis of novel conjugates and complexes for the delivery of molecules, including, but not limited to, small molecules, lipids, phospholipids, nucleosides, nucleotides, nucleic acids, antibodies, toxins, negatively charged polymers, and other polymers, for example proteins, peptides, hormones, carbohydrates, polyethylene glycols, or polyamines, across cellular membranes. In general, the transporters described are designed to be used either individually or as part of a multi-component system, with or without degradable linkers. These compounds are expected to improve delivery and/or localization of nucleic acid molecules of the presently disclosed subject matter into a number of cell types originating from different tissues, in the presence or absence of serum (see U.S. Pat. No. 5,854,038). Conjugates of the molecules described herein can be attached to biologically active molecules via linkers that are biodegradable, such as biodegradable nucleic acid linker molecules.

The term “biodegradable linker” as used herein, refers to a nucleic acid or non-nucleic acid linker molecule that is designed as a biodegradable linker to connect one molecule to another molecule, for example, a biologically active molecule to a siRNA molecule of the presently disclosed subject matter or the sense and antisense strands of a siRNA molecule of the presently disclosed subject matter. The biodegradable linker is designed such that its stability can be modulated for a particular purpose, such as delivery to a particular tissue or cell type. The stability of a nucleic acid-based biodegradable linker molecule can be modulated by using various chemistries, for example combinations of ribonucleotides, deoxyribonucleotides, and chemically modified nucleotides, such as 2′-O-methyl, 2′-fluoro, 2′-amino, 2′-O-amino, 2′-C-allyl, 2′-O-allyl, and other 2′-modified or base modified nucleotides. The biodegradable nucleic acid linker molecule can be a dimer, trimer, tetramer or longer nucleic acid molecule, for example, an oligonucleotide of about 2, 3, 4, 5, 6, 7, 8, 9, 10, 11, 12, 13, 14, 15, 16, 17, 18, 19, or 20 nucleotides in length, or can comprise a single nucleotide with a phosphorus-based linkage, for example, a phosphoramidate or phosphodiester linkage. The biodegradable nucleic acid linker molecule can also comprise nucleic acid backbone, nucleic acid sugar, or nucleic acid base modifications.

The term “biodegradable” as used herein, refers to degradation in a biological system, for example enzymatic degradation or chemical degradation.

The term “biologically active molecule” as used herein, refers to compounds or molecules that are capable of eliciting or modifying a biological response in a system. Non-limiting examples of biologically active siRNA molecules either alone or in combination with other molecules provided by the presently disclosed subject matter include therapeutically active molecules such as antibodies, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, siRNA, dsRNA, allozymes, aptamers, decoys, and analogs thereof. Biologically active molecules of the presently disclosed subject matter also include molecules capable of modulating the pharmacokinetics and/or pharmacodynamics of other biologically active molecules, for example, lipids and polymers such as polyamines, polyamides, polyethylene glycol, and other polyethers.

The term “phospholipid” as used herein, refers to a hydrophobic molecule comprising at least one phosphorus group. For example, a phospholipid can comprise a phosphorus-containing group and saturated or unsaturated alkyl group, optionally substituted with OH, COOH, oxo, amine, or substituted or unsubstituted aryl groups.

Nucleic acid molecules (e.g., siRNA molecules) delivered exogenously are intended to be stable within cells until the level of the target RNA has been reduced sufficiently. The nucleic acid molecules are resistant to nucleases in order to function as effective intracellular therapeutic agents. Improvements in the chemical synthesis of nucleic acid molecules described in the presently disclosed subject matter and in the art have expanded the ability to modify nucleic acid molecules by introducing nucleotide modifications to enhance their nuclease stability as described above.

In some embodiments, siRNA molecules having chemical modifications that maintain or enhance enzymatic activity of proteins involved in RNAi are provided. Such nucleic acids are also generally more resistant to nucleases than unmodified nucleic acids. Thus, in vitro and/or in vivo activity should not be significantly lowered.

Use of the nucleic acid-based molecules of the presently disclosed subject matter will lead to better treatment of the disease progression by affording the possibility of combination therapies (e.g., multiple siRNA molecules targeted to different genes; nucleic acid molecules coupled with known small molecule modulators; or intermittent treatment with combinations of molecules, including different motifs and/or other chemical or biological molecules). The treatment of subjects with siRNA molecules can also include combinations of different types of nucleic acid molecules, such as enzymatic nucleic acid molecules (ribozymes), allozymes, antisense, 2,5-A oligoadenylate, decoys, aptamers etc.

In another aspect a siRNA molecule of the presently disclosed subject matter comprises one or more 5′ and/or 3′-cap structures, for example on only the sense siRNA strand, antisense siRNA strand, or both siRNA strands.

As used herein, the phrase “cap structure” is meant to refer to chemical modifications that have been incorporated at either terminus of the oligonucleotide (see e.g., U.S. Pat. No. 5,998,203, incorporated by reference herein). These terminal modifications protect the nucleic acid molecule from exonuclease degradation, and can help in delivery and/or localization within a cell. The cap can be present at the 5′-terminus (5′-cap) or at the 3′-terminal (3′-cap), or can be present on both termini. In non-limiting examples: the 5′-cap is selected from the group comprising inverted abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

In some embodiments, the 3′-cap is selected from a group comprising 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (see generally Beaucage & Iyer, 1993; incorporated by reference herein).

As used herein, the term “non-nucleotide” refers to any group or compound which can be incorporated into a nucleic acid chain in the place of one or more nucleotide units, including either sugar and/or phosphate substitutions, and allows the remaining bases to exhibit their enzymatic activity. The group or compound is typically abasic, in that it does not typically contain a commonly recognized nucleotide base, such as adenine (A), guanine (G), cytosine (C), thymine (T), or uracil (U), and therefore lacks a base at the 1′-position.

As used herein, the term “alkyl” group refers to a saturated aliphatic hydrocarbon, including straight-chain, branched-chain, and cyclic alkyl groups. In some embodiments, the alkyl group has 1 to 12 carbons. In some embodiments, it is a lower alkyl of from 1 to 7 carbons, and in some embodiments it is a lower alkyl of from 1 to 4 carbons. The alkyl group can be substituted or unsubstituted. When substituted the substituted group(s) is in alternative embodiments, hydroxyl, cyano, alkoxy, ═O, ═S, NO₂ or N(CH₃)₂, amino, or SH.

The term “alkyl” also includes alkenyl groups that are unsaturated hydrocarbon groups containing at least one carbon-carbon double bond, including straight-chain, branched-chain, and cyclic groups. In some embodiments, the alkenyl group has 1 to 12 carbons. In some embodiments, it is a lower alkenyl of from 1 to 7 carbons, and in some embodiments it is a lower alkenyl of from 1 to 4 carbons. The alkenyl group can be substituted or unsubstituted. When substituted the substituted group(s) is in alternative embodiments, hydroxyl, cyano, alkoxy, ═O, ═S, NO₂, halogen, N(CH₃)₂, amino, or SH.

The term “alkyl” also includes alkynyl groups that have an unsaturated hydrocarbon group containing at least one carbon-carbon triple bond, including straight-chain, branched-chain, and cyclic groups. In some embodiments, the alkynyl group has 1 to 12 carbons. In some embodiments, it is a lower alkynyl of from 1 to 7 carbons, and in some embodiments it is a lower alkynyl of from 1 to 4 carbons. The alkynyl group can be substituted or unsubstituted. When substituted the substituted group(s) is in alternative embodiments, hydroxyl, cyano, alkoxy, ═O, ═S, NO₂ or N(CH₃)₂, amino or SH.

Such alkyl groups can also include aryl, alkylaryl, carbocyclic aryl, heterocyclic aryl, amide, and ester groups. An “aryl” group refers to an aromatic group that has at least one ring having a conjugated pi electron system and includes carbocyclic aryl, heterocyclic aryl, and biaryl groups, all of which can be optionally substituted. Exemplary substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazoyl and the like, all optionally substituted. An “amide” refers to an —C(O)—NH—R, where R is either alkyl, aryl, alkylaryl, or hydrogen. An “ester” refers to an C(O)OR′, where R is either alkyl, aryl, alkylaryl, or hydrogen.

The term “nucleotide” is used herein as recognized in the art to include natural bases (standard), and modified bases well known in the art. Such bases are generally located at the 1′ position of a nucleotide sugar moiety. Nucleotides generally comprise a base, sugar, and a phosphate group. The nucleotides can be unmodified or modified at the sugar, phosphate, and/or base moiety, (also referred to interchangeably as nucleotide analogs, modified nucleotides, non-natural nucleotides, non-standard nucleotides, and other; see e.g., Usman et al., 1996; PCT International Publication Nos. WO 92/07065 and WO 93/15187, all incorporated by reference herein). There are several examples of modified nucleic acid bases known in the art as summarized by Limbach et al., 1994. Some of the non-limiting examples of base modifications that can be introduced into nucleic acid molecules include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine), 6-azapyrimidines and 6-alkylpyrimidines (e.g. 6-methyluridine), propyne, and others (Burgin et al., 1996; Uhlman & Peyman, 1990). By “modified bases” in this aspect is meant nucleotide bases other than adenine, guanine, cytosine, and uracil at 1′ position or their equivalents.

In some embodiments, the presently disclosed subject matter features modified siRNA molecules, with phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker & Leumann, 1995 and De Mesmaeker et al., 1994.

As used herein, the term “abasic” refers to sugar moieties lacking a commonly recognized nucleoside base (e.g., A, C, G, T, or U) or having 2005/032486 PCT/US2004/032710 other chemical groups in place of the commonly recognized base at the 1′ position. See e.g., U.S. Pat. No. 5,998,203.

As used herein, the phrase “unmodified nucleoside” refers to one of the bases adenine, cytosine, guanine, thymine, or uracil joined to the 1′ carbon of β-D-ribo-furanose.

By “modified nucleoside” is meant any nucleotide base which contains a modification in the chemical structure of an unmodified nucleotide base, sugar and/or phosphate.

In connection with 2′-modified nucleotides as described for the presently disclosed subject matter, by “amino” is meant 2′—NH₂ or 2′-O—NH₂, which can be modified or unmodified. Such modified groups are described, for example, in U.S. Pat. Nos. 5,672,695 and 6,248,878, which are both incorporated by reference in their entireties.

Various modifications to nucleic acid siRNA structure can be made to enhance the utility of these molecules. Such modifications will enhance shelf-life, half-life in vitro, stability, and/or ease of introduction of such oligonucleotides to the target site (for example, to enhance penetration of cellular membranes, and confer the ability to recognize and bind to targeted cells).

IV. Vectors

In another aspect of the presently disclosed subject matter, siRNA molecules are expressed from transcription units inserted into nucleic acid vectors (alternatively referred to generally as “recombinant vectors” or “expression vectors”). The recombinant vectors can be, for example, DNA plasmids or viral vectors. Various expression vectors are known in the art. The selection of the appropriate expression vector can be made on the basis of several factors including, but not limited to the cell type wherein expression is desired. For example, mammalian expression vectors can be used to express the nucleic acids of the presently disclosed subject matter when the hypoxic cell is a mammalian cell.

Exemplary siRNA expressing viral vectors can be constructed based on adenovirus, adeno-associated virus, retrovirus, or alphavirus. The recombinant vectors capable of expressing the siRNA molecules can be 2005/032486 PCT/US2004/032710 delivered as described herein, and persist in target cells. Alternatively, viral vectors can be used that provide for transient expression of siRNA molecules. In some embodiments, a vector of the presently disclosed subject matter is an adenovirus vector.

Incorporation of a nucleic acid construct into a viral genome can be optionally performed by ligating the construct into an appropriate restriction site in the genome of the virus. Viral genomes can then be packaged into viral coats or capsids by any suitable procedure. In particular, any suitable packaging cell line can be used to generate viral vectors of the presently disclosed subject matter. These packaging lines complement the conditionally replication deficient viral genomes of the presently disclosed subject matter, as they include, typically incorporated into their genomes, the genes which have been put under an inducible promoter deleted in the conditionally replication competent vectors. Thus, the use of packaging lines allows viral vectors of the presently disclosed subject matter to be generated in culture.

V. Applications

The presently disclosed subject matter provides a method for inhibiting expression of a hypoxia-inducible gene in a subject, the method comprising (a) providing a subject containing a target cell, wherein the target cell comprises the hypoxia-inducible gene and the hypoxia-inducible gene is expressed in the target cell when the target cell is exposed to hypoxic conditions; and (b) introducing the ribonucleic acid (RNA) into the target cell. In some embodiments, the hypoxia-inducible gene is HIF-1α, for example human HIF-1α (SEQ ID NO: 1) or mouse HIF-1α (SEQ ID NO: 3), although the HIF-1α gene from other species can be targeted using the methods and compositions disclosed herein. For example, siRNAs can be designed using the methods disclosed herein to target HIF-1α mRNAs from Bos taurus, Rattus norvegicus, Sus scrofa, or Canis familiaris using the nucleotide sequence information available at GENBANK® Accession Nos. NM_{—}174339, NM_{—}024359, AJ439692, and AJ439691, respectively.

The presently disclosed subject matter also provides methods for suppressing the growth of a hypoxia cell in a subject. In some embodiments, the method comprises contacting the cell with a vector comprising an siRNA molecule under conditions sufficient to allow entry of the vector into the cell. An siRNA molecule can comprise a sense region and an antisense region, wherein the antisense region comprises a nucleic acid sequence complementary to an RNA sequence encoding a hypoxia-inducible gene product and the sense region comprises a nucleic acid sequence complementary to the antisense region.

In some embodiments, the method comprises contacting a hypoxic cell in a tumor with a vector encoding an siRNA under conditions sufficient to allow entry of the vector into the cell. In some embodiments of the present method, the vector is an adenovirus vector. For example, the disclosed adenovirus vectors can be useful in the treatment of both primary and metastatic solid tumors and carcinomas of the breast; colon; rectum; lung; oropharynx; hypopharynx; esophagus; stomach; pancreas; liver; gallbladder; bile ducts; small intestine; urinary tract including kidney, bladder and urothelium; female genital tract including cervix, uterus, ovaries, choriocarcinoma and gestational trophoblastic disease; male genital tract including prostate, seminal vesicles, testes and germ cell tumors; endocrine glands including thyroid, adrenal, and pituitary; skin including hemangiomas, melanomas, sarcomas arising from bone or soft tissues and Kaposi's sarcoma; tumors of the brain, nerves, eyes, and meninges including astrocytomas, gliomas, glioblastomas, retinoblastomas, neuromas, neuroblastomas, Schwannomas and meningiomas; solid tumors arising from hematopoietic malignancies such as leukemias and including chloromas, plasmacytomas, plaques and tumors of mycosis fungoides and cutaneous T-cell lymphoma/leukemia; lymphomas including both Hodgkin's and non-Hodgkin's lymphomas.

The compositions of the presently disclosed subject matter can also be useful for the prevention of metastases from the tumors described above either when used alone or in combination with radiotherapeutic, photodynamic, and/or chemotherapeutic treatments conventionally administered to patients for treating disorders, including angiogenic disorders. For example, a tumor can be treated conventionally with surgery, photodynamic therapy, radiation and/or chemotherapy followed by administration of the compositions of the presently disclosed subject matter to extend the dormancy of micrometastases and to stabilize and inhibit the growth of any residual primary tumor. Indeed, virus administration can be provided before, during, or after radiotherapy; before, during, or after chemotherapy; and/or before, during, or after photodynamic therapy.

The compositions and methods of the presently disclosed subject matter are not limited to use in cells that have elevated HIF-1 expression due to hypoxia. They can also be used in any cell in which inappropriate HIF-1 activity results in the expression of hypoxia-inducible genes. For example, loss of pVHL function has been reported in a familial angiomatous syndrome, and also in the majority of sporadic central nervous system hemangioblastomas and clear cell renal carcinomas (reviewed in Ivan & Kaelin, 2001). Furthermore, pVHL mutations that have been associated with renal cell carcinoma and/or hemangioblastomas have all been shown to interfere with pVHL's ability to regulate HIF-1α activity (Maxwell et al., 2001). Thus, the compositions and methods of the presently disclosed subject matter are applicable to cells that have lost pVHL function.

In addition, a recent report suggested that HIF-1 accumulates in some tumor cells even under normoxic conditions. It has long been known that some cancer cells display high rates of glycolysis under aerobic conditions, a phenomenon known as the Warburg effect. Evidence suggests that the Warburg effect is characterized by the accumulation of HIF-1 in transformed cells in normoxic areas of tumors, leading to glycolysis under aerobic conditions. Further, the induction of HIF-1 in these cells appears to be mediated by the pp60^{c-Src} protein (see Karni et al., 2002), which has been implicated in several forms of human cancer (reviewed in Brickell, 1992). Thus, the compositions and methods of the presently disclosed subject matter are applicable to cells that have elevated pp60^{c-Src} activity.

In some embodiments, then, the elevation of pp60^{c-Src} or the loss of VHL function therefore allows the HIF-1α-directed siRNA containing adenovirus vectors to cause a down regulation of HIF-1 activity in tumor cells (for example, those derived from VHL-deficient clear cell renal carcinomas) in the absence of hypoxia. Under these circumstances, every tumor cell is targeted as HIF-1 is activated in every cell.

A hypoxia inducible promoter of the presently disclosed subject matter can further be responsive to non-hypoxia stimuli that can be used in combined therapy treatments as disclosed herein. For example, the mortalin promoter is induced by low doses of ionizing radiation (Sadekova et al., 1997), the hsp27 promoter is activated by 17β-estradiol and estrogen receptor agonists (Porter et al., 2001), the HLA-G promoter is induced by arsenite, and hsp promoters can be activated by photodynamic therapy (Luna et al., 2000). Thus, an siRNA encoded by a vector (for example, an adenovirus vector) can be operatively linked to one of these promoters or additional DNA elements that support combined therapy treatments. Virus administration can be provided before, during, or after radiotherapy; before, during, or after chemotherapy; and/or before, during, or after photodynamic therapy.

V.A. Subjects

The subject treated in the presently disclosed subject matter in its many embodiments is desirably a human subject, although it is to be understood that the principles of the presently disclosed subject matter indicate that the presently disclosed subject matter is effective with respect to invertebrate and to all vertebrate species, including mammals, which are intended to be included in the term “subject”. Moreover, a mammal is understood to include any mammalian species in which treatment or prevention of cancer is desirable, particularly agricultural and domestic mammalian species.

The methods of the presently disclosed subject matter are particularly useful in the treatment of warm-blooded vertebrates. Thus, the presently disclosed subject matter concerns mammals and birds.

More particularly provided is the treatment of mammals such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economic importance to humans. Thus, contemplated is the treatment of livestock, including, but not limited to, domesticated swine (pigs and hogs), ruminants, horses, poultry, and the like.

V.B. Formulation

The adenovirus vectors of the presently disclosed subject matter comprise in some embodiments a composition that includes a pharmaceutically acceptable carrier. Any suitable pharmaceutical formulation can be used to prepare the adenovirus vectors for administration to a subject.

For example, suitable formulations can include aqueous and non-aqueous sterile injection solutions which can contain anti-oxidants, buffers, bacteriostats, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are SDS, in one example in the range of 0.1 to 10 mg/ml, in another example about 2.0 mg/ml; and/or mannitol or another sugar, for example in the range of 10 to 100 mg/ml, in another example about 30 mg/ml; and/or phosphate-buffered saline (PBS).

It should be understood that in addition to the ingredients particularly mentioned above the formulations of this presently disclosed subject matter can include other agents conventional in the art having regard to the type of formulation in question. For example, sterile pyrogen-free aqueous and non-aqueous solutions can be used.

The therapeutic regimens and compositions of the presently disclosed subject matter can be used with additional adjuvants or biological response modifiers including, but not limited to, the cytokines IFN-α, IFN-γ, IL2, IL4, IL6, TNF, or other cytokine affecting immune cells. In accordance with this aspect of the presently disclosed subject matter, the disclosed nucleic acid molecules can be administered in combination therapy with one or more of these cytokines.

V.C. Administration

Administration of the compositions of the presently disclosed subject matter can be by any method known to one of ordinary skill in the art, including, but not limited to intravenous administration, intrasynovial administration, transdermal administration, intramuscular administration, subcutaneous administration, topical administration, rectal administration, intravaginal administration, intratumoral administration, oral administration, buccal administration, nasal administration, parenteral administration, inhalation, and insufflation. In some embodiments, suitable methods for administration of a nucleic acid molecule of the presently disclosed subject matter (for example, using an adenovirus vector) include but are not limited to intravenous or intratumoral injection. Alternatively, a nucleic acid molecule can be deposited at a site in need of treatment in any other manner, for example by spraying a composition comprising a nucleic acid molecule within the pulmonary pathways. The particular mode of administering a composition of the presently disclosed subject matter depends on various factors, including the distribution and abundance of cells to be treated, the vector employed, additional tissue- or cell-targeting features of the vector, and mechanisms for metabolism or removal of the vector from its site of administration. For example, relatively superficial tumors can be injected intratumorally. By contrast, internal tumors can be treated by intravenous injection.

In some embodiments, the method of administration encompasses features for regionalized vector delivery or accumulation at the site in need of treatment. In one example, an adenovirus vector is delivered intratumorally. In some embodiments, selective delivery of a adenovirus vector to a tumor is accomplished by intravenous injection of the construct

For delivery of adenovirus vectors to pulmonary pathways, adenovirus vectors of the presently disclosed subject matter can be formulated as an aerosol or coarse spray. Methods for preparation and administration of aerosol or spray formulations can be found, for example, in Cipolla et al., 2000, and in U.S. Pat. Nos. 5,858,784; 6,013,638; 6,022,737; and 6,136,295.

V.D. Dose

An effective dose of a composition of the presently disclosed subject matter is administered to a subject in need thereof. A “therapeutically effective amount” is an amount of the composition sufficient to produce a measurable response (e.g., a cytolytic response in a subject being treated). In some embodiments, an activity that inhibits tumor growth is measured. Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the therapeutic composition, the route of administration, combination with other drugs or treatments, the severity of the condition being treated, and the condition and prior medical history of the subject being treated. However, it is within the skill of the art to start doses of the compound at levels lower than required to achieve the desired therapeutic effect and to gradually increase the dosage until the desired effect is achieved.

The potency of a composition can vary, and therefore a “therapeutically effective” amount can vary. However, using the assay methods described herein below, one skilled in the art can readily assess the potency and efficacy of a candidate modulator of this presently disclosed subject matter and adjust the therapeutic regimen accordingly.

After review of the disclosure of the presently disclosed subject matter presented herein, one of ordinary skill in the art can tailor the dosages to an individual patient, taking into account the particular formulation, method of administration to be used with the composition, and tumor size. Further calculations of dose can consider patient height and weight, severity and stage of symptoms, and the presence of additional deleterious physical conditions. Such adjustments or variations, as well as evaluation of when and how to make such adjustments or variations, are well known to those of ordinary skill in the art of medicine.

For example, for local administration of viral vectors, previous clinical studies have demonstrated that up to 10¹³ plaque-forming units (pfu) of virus can be injected with minimal toxicity. In human patients, 1×10⁹-1×10¹³ pfu are routinely used (see Habib et al., 1999). To determine an appropriate dose within this range, preliminary treatments can begin with 1×10⁹ pfu, and the dose level can be escalated in the absence of dose-limiting toxicity. Toxicity can be assessed using criteria set forth by the National Cancer Institute and is reasonably defined as any grade 4 toxicity or any grade 3 toxicity persisting more than 1 week. Dose is also modified to maximize anti-tumor or anti-angiogenic activity. Representative criteria and methods for assessing anti-tumor and/or anti-angiogenic activity are described herein below. With replicative virus vectors, a dosage of about 1×10⁷ to 1×10⁸ pfu can be used in some instances.

An adenovirus construct as disclosed herein can be packaged into adenovirus vectors and the prepared virus titer reaches at least 1×10⁶-1×10⁷ pfu/ml. The adenoviral construct is administered in the amount of 1.0 pfu/target cell. Thus, administration of a minimal level of adenoviral construct to thereby provide a therapeutic level of an siRNA encoded by the adenovirus vector comprises an aspect of the presently disclosed subject matter.

### EXAMPLES

The following Examples have been included to illustrate modes of the presently disclosed subject matter. Certain aspects of the following Examples are described in terms of techniques and procedures found or contemplated by the present co-inventors to work well in the practice of the presently disclosed subject matter. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### Identification of Potential siRNA Target Sites in Any RNA Sequence

The sequence of an RNA target of interest, such as a human mRNA transcript, is screened for target sites, for example by using a computer-based folding algorithm. In a non-limiting example, the sequence of a gene or RNA gene transcript derived from a database, such as GENBANK®, is used to generate siRNA targets having complementarity to the target. Such sequences can be obtained from a database, or can be determined experimentally as known in the art. Target sites that are known, for example, those target sites determined to be effective target sites based on studies with other nucleic acid molecules, for example ribozymes or antisense, or those targets known to be associated with a disease or condition such as those sites containing mutations or deletions, can be used to design siRNA molecules targeting those sites as well. Various parameters can be used to determine which sites are the most suitable target sites within the target RNA sequence. These parameters include but are not limited to secondary or tertiary RNA structure, the nucleotide base composition of the target sequence, the degree of homology between various regions of the target sequence, or the relative position of the target sequence within the RNA transcript. Based on these determinations, any number of target sites within the RNA transcript can be chosen to screen siRNA molecules for efficacy, for example by using in vitro RNA cleavage assays, cell culture, or animal models. In a non-limiting example, anywhere from 1 to 1000 target sites are chosen within the transcript based on the size of the siRNA construct to be used. High throughput screening assays can be developed for screening siRNA molecules using methods known in the art, such as with multi-well or multi-plate assays to determine efficient reduction in target gene expression.

### Example 2

### Tandem Synthesis of siRNA Constructs

Exemplary siRNA molecules of the presently disclosed subject matter are synthesized in tandem using a cleavable linker, for example a succinyl-based linker. Tandem synthesis as described herein is followed by a one step purification process that provides RNAi molecules in high yield. This approach is highly amenable to siRNA synthesis in support of high throughput RNAi screening, and can be readily adapted to multi-column or multi-well synthesis platforms.

After completing a tandem synthesis of an siRNA oligo and its complement in which the 5′-terminal dimethoxytrityl (5′-O-DMT) group remains intact (trityl on synthesis), the oligonucleotides are deprotected as described above. Following deprotection, the siRNA sequence strands are allowed to spontaneously hybridize. This hybridization yields a duplex in which one strand has retained the 5′-O-DMT group while the complementary strand comprises a terminal 5′-hydroxyl. The newly formed duplex to behaves as a single molecule during routine solid-phase extraction purification (Trityl-On purification) even though only one molecule has a dimethoxytrityl group. Because the strands form a stable duplex, this dimethoxytrityl group (or an equivalent group, such as other trityl groups or other hydrophobic moieties) is all that is required to purify the pair of oligos, for example by using a C18 cartridge.

Standard phosphoramidite synthesis chemistry is used up to point of introducing a tandem linker, such as an inverted deoxyabasic succinate linker or an equivalent cleavable linker. A non-limiting example of linker coupling conditions that can be used includes a hindered base such as diisopropylethylamine (DIPA) and/or DMAP in the presence of an activator reagent such as bromotripyrrolidinophosphoniumhexafluororophosphate (PyBrOP). After the linker is coupled, standard synthesis chemistry is utilized to complete synthesis of the second sequence leaving the terminal the 5′-O-DMT intact. Following synthesis, the resulting oligonucleotide is deprotected according to the procedures described herein and quenched with a suitable buffer, for example with 50 mM sodium acetate (NaOAc) or 1.5 M NH₄H₂CO₃.

Purification of the siRNA duplex can be readily accomplished using solid phase extraction, for example using a C18 SEPPAK® 1 g cartridge (Waters Corp., Milford, Mass., United States of America) conditioned with 1 column volume (CV) of acetonitrile, 2 CV H₂O, and 2 CV 50 mM NaOAc. The sample is loaded and then washed with 1 CV H₂O or 50 mM NaOAc. Failure sequences are eluted with 1 CV 14% ACN (Aqueous with 50 mM NaOAc and 50 mM NaCl). The column is then washed, for example with 1 CV H₂O followed by on-column detritylation, for example by passing 1 CV of 1% aqueous trifluoroacetic acid (TFA) over the column, then adding a second CV of 1% aqueous TFA to the column and allowing to stand for approx. 10 minutes. The remaining TFA solution is removed and the column washed with H₂O followed by 1 CV 1M NaCl and additional H₂O. The siRNA duplex product is then eluted, for example using 1 CV 20% aqueous ACN.

### Example 3

### Chemical Synthesis and Purification of siRNA

siRNA molecules can be designed to interact with various sites in the RNA message, for example target sequences within the RNA sequences described herein. The sequence of one strand of the siRNA molecule(s) are complementary to the target site sequences described above. The siRNA molecules can be chemically synthesized using methods described herein. Inactive siRNA molecules that are used as control sequences can be synthesized by scrambling the sequence of the siRNA molecules such that it is not complementary to the target sequence.

### Example 4

### Cell Culture

The following cell lines were used: HEK 293 (hereinafter “293 cells”), an adenovirus E1 gene transduced human embryonic kidney cell line; HeLa, a human cervical adenocarcinoma cell line obtained from American Type Culture Collection (ATCC; Manassas, Va., United States of America), and HCT116, a human colon cancer cell line obtained from the Tissue Culture Facility at Duke University Medical Center (Durham, N.C., USA; also available from the ATCC). Cells were cultured in Dulbecco's modified Eagle's medium (DMEM; Invitrogen Corp., Carlsbad, Calif., United States of America) with 10% fetal bovine serum (FBS), 100 units/ml penicillin, and 100 μg/ml streptomycin at 37° C., 5% CO₂.

### Example 5

### Designing siRNA-encoding Minigenes Targeted to HIF-1α

To design the siRNA-encoding minigenes, an Internet-based program available at the website of Ambion Inc. (Austin, Tex., United States of America) was used. Oligonucleotide DNA sequences based on these targeting sequences were then synthesized by commercial sources. These oligos contain two 19-mer complementary targeting sequences with a loop sequence separating them and a polythymidine tract to terminate transcription. In addition, the oligos were engineered to possess Bam HI- and Hind III-compatible overhangs to facilitate ligation of the oligos into the expression vector pSILENCER™ 2.0 (Ambion Inc., Austin, Tex., United States of America), which is a plasmid with a human U6 gene-based RNA polymerase III promoter. The derived HIF-1α-targeted minigene-encoding plasmid was pSilencer-siHIF-1α. The control plasmid was pSilencer-siNT (obtained from Ambion Inc.), which is a plasmid with a similar structure but encoding a nonsense minigene with no homology to any known sequence in the human genome. The sequence of the control minigene is MT TCT CCG MC GTG TCA CGT (SEQ ID NO: 8).

### Example 6

### Adenovirus Production

The ADEASYT™ system of adenovirus packaging, including plasmids pAdtrack and pAdeasy-1, and Escherichia coli BJ5183 cells, is commercially available from Stratagene Corporation (La Jolla, Calif., United States of America). The siRNA-encoding gene expression cassette (with the U6 gene promoter) was then excised from pSilencer-siHIF-1α and subcloned into the EcoR V/Hind III sites of pAdTrack using Pvu II and Hind III. The resulting plasmid was called pAdTrack-siHIF-1α. Packaging and production of the adenovirus that carries the HIF-1α-targeted siRNA gene was carried out using previously described approaches (He et al., 1998). Briefly, the pAdtrack-U6-siHIF-1a plasmid was linearized by Pme I and recombined with the pAdeasy-1 plasmid in the recA⁺ bacterial strain BJ5183. The resulting plasmid, pAdeasy-siHIF-1α, was transfected into low passage (less than 30 passages) 293 cells after linearization of the plasmid with Pac I. After 7-10 days, an infectious adenovirus vector, AdsiHIF-1α, was obtained. Large-scale preparation of the particles was carried out subsequently according to established protocols (He et al., 1998).

### Example 7

### Quantitative PCR Assessment of HIF-1α Messenger RNA Levels

To measure the level of HIF-1α mRNA in cells that have been infected with the siRNA-encoding adenovirus vectors, quantitative PCR (Q-PCR) technology was used. Twenty-four hours after infection by adenovirus vectors (AdsiNT or AdsiHIF-1α), total RNA from the infected cells were extracted by use of the RNEASY® kit (QIAGEN Inc., Valencia, Calif., United States of America). Afterwards, cDNA from the mRNA were synthesized by use of the SUPERSCRIPT™ first-strand synthesis system for RT-PCR (Invitrogen Corp., Carlsbad, Calif., United States of America). The generated cDNA was then used as templates in Q-PCR reactions. The Q-PCR reactions were carried out by use of the QUANTITECH™ SYBR® Green PCR kit (QIAGEN Inc.) in an ABI PRISM® 7900 apparatus (Applied Biosystems, Foster City, Calif., United States of America). Relative quantifications of HIF-1α were performed by a comparative CT method. The relative amount of target (HIF-1α), normalized to an endogenous sequence, is given by 2-DD CT.

The primers used for the amplification of β-actin were: 5′-TCAAGATCATTGCTCCTCCTG-3′ (forward primer; SEQ ID NO: 9) and 5′-CTGCTTGCTGATCCACATCTG-3′ (reverse primer; SEQ ID NO: 10). The primers used for the amplification of the HIF1α gene were: 5′-CTGATCATCTGACCAAAACTC-3′ (forward primer; SEQ ID NO: 11) and 5′-GTTTCAACCCAGACATATCCAC-3′ (reverse primer; SEQ ID NO: 12).

### Example 8

### Hypoxia Treatment

Hypoxia treatment of cells was achieved by incubating cells in a Bactron Anaerobic/Environmental Chamber (Sheldon Manufacturing, Corvallis, Oreg., United States of America). During incubation, a humidified environment at 37° C. was maintained. In addition, the atmosphere was maintained at 5% CO₂ and 0.5% O₂.

### Example 9

### Western Blot Analysis

Antibodies to caspase-3, Bcl-XL, and HIF-1α were purchased from Cell Signaling Technology (Beverly, Mass., United States of America), BD PharMingen (Palo Alto, Calif., United States of America), and Santa Cruz Biotechnology (Santa Cruz, Calif., United States of America), respectively. HeLa cells were infected with adenovirus and subjected to hypoxia treatment. Treated cells were collected and lysed. About 0.6 μg to 2 μg of total protein from the cell lysates were separated by electrophoresis on a 6% sodium dodecyl sulfate polyacrylamide gel electrophoresis (SDS-PAGE) gel. The proteins were then transferred to a nitrocellulose membrane using an electroblotting device. The membrane was blocked with 5% non-fat milk in phosphate-buffered saline plus 0.1% Tween 20 (PBST) overnight at 4° C. After overnight blocking, the membranes were incubated with the primary antibody for 2 hours, washed with PBST 3 times (15 minutes each time), and then incubated with a horseradish peroxidase (HRP)-conjugated secondary antibody (IgG). After incubation with the secondary antibody, the membrane washed with PBST for 3 times. Positive binding was visualized by chemiluminescence using the ECL™ kit (Amersham, Arlington Heights, Ill., United States of America).

### Example 10

### Hochest 33342 Staining for Apoptotic Cells

HeLa cells cultured in 12-well plates to 60-70% confluence were infected with AdsiHIF-1 or AdsiNT at a multiplicity of infection (m.o.i.) of 10 for 24 hours, and then subjected to hypoxia (0.5% O₂) for 24 hours. Hypoxia was induced by placing the cells in an anaerobic tissue culture hood (Sheldon Manufacturing, Corvallis, Oreg., United States of America) maintained at 37° C. At the end of the hypoxia treatment, the cells were fixed in methanol:acetic acid (3:1) for 5 minutes at 4° C. and washed with sterile distilled water three times. The cells were then stained with Hoechst 33342 (5 μg/ml; Hoechst, Germany) for 10 minutes at room temperature. After washing the cells as before, the fraction of apoptotic non-apoptotic cells was determined by counting the cells under a fluorescence microscope. Four randomly chosen areas were counted and averaged to derive a value for the apoptotic cell fraction. Counting was carried out by two independent investigators.

### Example 11

### Tumor Growth Delay

About 5×10⁶ cells HeLa cells (in 50 μl) infected with AdsiHIF-1α or AdsiNT viruses (m.o.i. 10) were transplanted subcutaneously in 50 μl of PBS into the right hind limbs of BALB/c nude mice 24 hours after virus infection. Each treatment group consisted of 8-10 animals. Growth curves were plotted as the mean relative treatment group tumor volume±standard error (SE). The following formula was used to calculate tumor volume:
V=(W²×L)/2
where W is the length of the shortest dimension and L is the length of the longest dimension. See Zhang et al., 2003.

In the second series of experiments, about 5×10⁶ HCT116 tumor cells were injected subcutaneously (in 50 μl of PBS) into the hind leg of nude mice. When tumors grew to sizes of 7-8 mm in diameter, adenovirus vectors were injected into the tumor mass (1×10⁸ pfu in 30 μl). About 24 hours later, the tumors were irradiated with a 4 MeV linear accelerator (Varian Medical Systems, Inc., Palo Alto, Calif., United States of America) at a dose rate of 2 Gray/min. Three doses were given at 6 Gy each. Adenovirus vectors were administered 24 hours prior to each dose. Tumor growth was then followed by daily measurement. Growth curves are plotted as the mean relative treatment group tumor volume±standard error of the mean (SEM).

Mean times to reaching three times initial tumor volumes (phase of exponential regrowth) for each group were calculated and compared using the Kruskal-Wallis and the two sided Mann Whitney tests (non-parametric).

### Discussion of Examples 4-11

### Down-regulation of HIF-1α in HeLa cells by Adenovirus Delivered siRNA

To target human HIF-1α using the siRNA-based approach, several siRNA targeting sequences were designed. Each siRNA was synthesized as complementary oligonucleotides and cloned into a pSILENCER™ 2.0 vector as described elsewhere (see Brummelkamp et al., 2002; Miyagishi & Taira, 2002; Yu et al., 2002). The resulting constructs were verified by sequencing and screened for their ability to down regulate HIF-1α expression in HeLa cells. The siRNA-encoding vector that was most effective appeared to be one targeted to nucleotide 244-262 downstream of the AUG start codon of the HIF-1 gene (GENBANK® Accession No. NM_{—}001530).

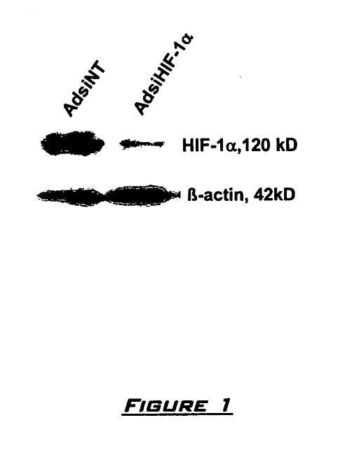

To further study the efficacy of the HIF-1α-targeted siRNA, the above siRNA encoding gene expression cassette was transferred into an adenovirus vector. This vector was then tested for its capacity to down-regulate HIF-1α in HeLa cells. After AdsiHIF-1α and AdsiNT (a control vector with a nonsense siRNA minigene) infection for 24 hours, HeLa cells were subjected to hypoxia (0.5% oxygen) for 24 hours. The cells were then harvested and western blot analysis was conducted. Results show that HIF-1α was greatly down regulated in AdsiHIF-1α-infected hypoxic HeLa cells (greater than 90%) but not in the control cells (see FIG. 1). In addition, quantitative PCR was used to measure the level of mRNA in AdsiNT and AdsiHIF-1α infected HeLa cells (under normoxic condition). Quantitative PCR indicated that the mRNA level of HIF-1α was reduced about 90% while the level of β-actin mRNA remained unchanged. These results thus indicated that an adenovirus vector can be a useful tool for delivery of siRNA into tumor cells.

### Sensitization of Tumor Cells to Hypoxia-Induced Cell Apoptosis by Adenovirus Delivered HIF-1a Targeted siRNA

Exposure to hypoxic conditions is known to induce apoptosis in many cells. However, the role of HIF-1α is controversial in this process. Some reports suggest that HIF-1α is a mediator of hypoxia-induced cell death (Dai et al., 2003). In support of this viewpoint is the observation that HIF-1α can activate the transcription of many pro-apoptotic genes, including NIX and NIP3 (Sowter et al., 2001). Other reports suggest that elevated HIF-1α expression could render tumor cells resistant to hypoxic exposure.

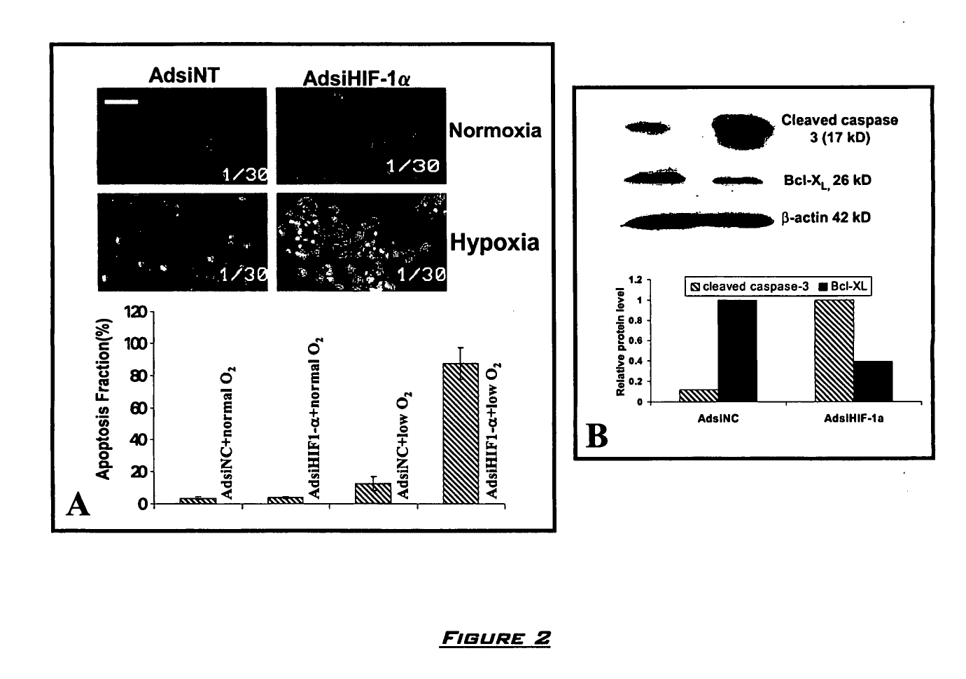

The effects of HIF-1α down regulation were examined in HeLa cells. HeLa cells were infected with adenovirus vector AdsiHIF-1α. Infection of cells with this vector has been shown to result in greater than 90% down regulation of HIF-1α at the mRNA and protein levels (see FIG. 1). After infecting HeLa cells with AdsiHIF-1α or AdsiNT vectors for 24 hours (each at an m.o.i. of 10), the cells were exposed to hypoxic conditions (0.5% O₂) for 24 hours. Hoechst 33342 nuclear staining was then used to quantify apoptosis in HeLa cells. Apoptotic cells were typically identified as those cells that possess significantly smaller, condensed, and fragmented nuclei under a fluorescence microscope (see FIG. 2A). In the AdsiHIF-1α-infected HeLa cells, approximately 87.3±9.7% cells were undergoing apoptotic cell death. This is compared with a 12.7±4.3% death rate in the control virus infected cells. Under normoxic conditions, negligible cell death was observed in either AdsiHIF-1α or control virus infected HeLa cells. Thus, the down regulation of HIF-1α can significantly enhance apoptosis in HeLa cells exposed to hypoxic conditions.

To determine the molecular mechanism underlying hypoxia-induced apoptotic cell death in HeLa cells, the levels of two proteins known to be involved in cellular apoptosis were also analyzed. These proteins include cleaved caspase-3, an effecter of apoptosis, and Bcl-X_{L}, a negative regulator of apoptosis. In AdsiHIF-1α-infected HeLa cells, hypoxia treatment caused a significant increase in the cleavage of caspase-3, indicating its activation. At the same time, hypoxia significantly reduced the expression of Bcl-X_{L} (see FIG. 2B), a cellular survival factor. These results provide strong evidence that inhibiting HIF-1α gene expression levels sensitizes tumor cells to hypoxia via activation of cellular apoptotic pathways.

### Anti-Tumor Effect of Silencing HF-1a Expression

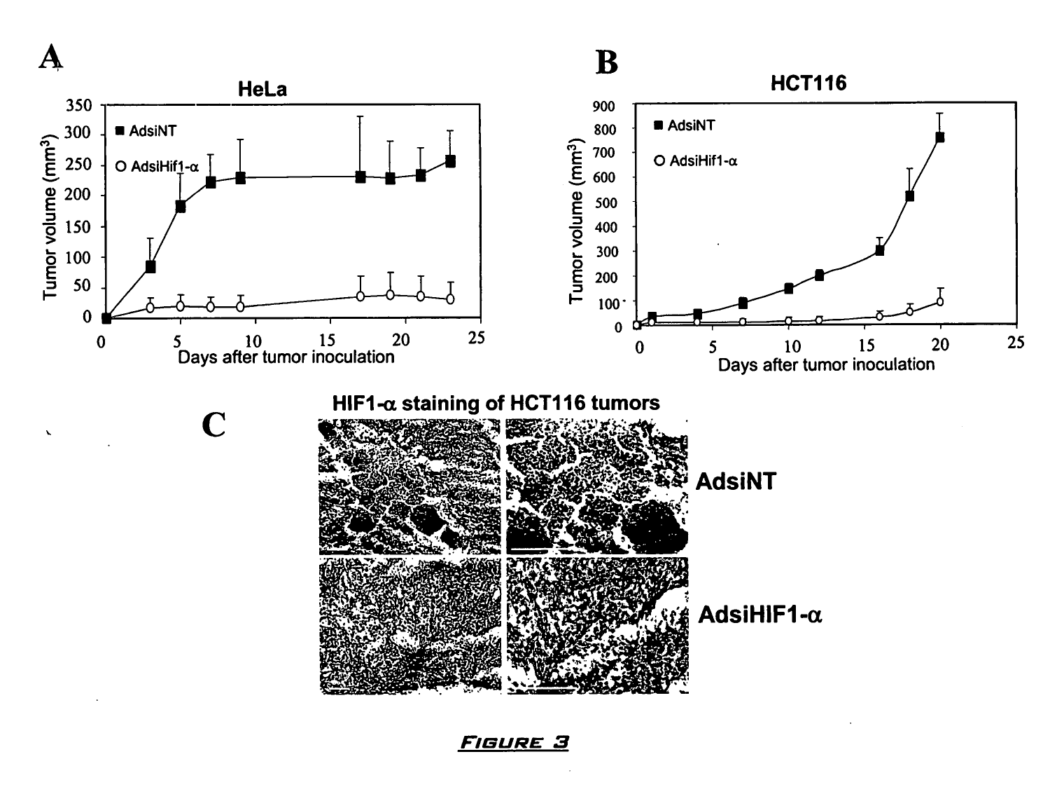

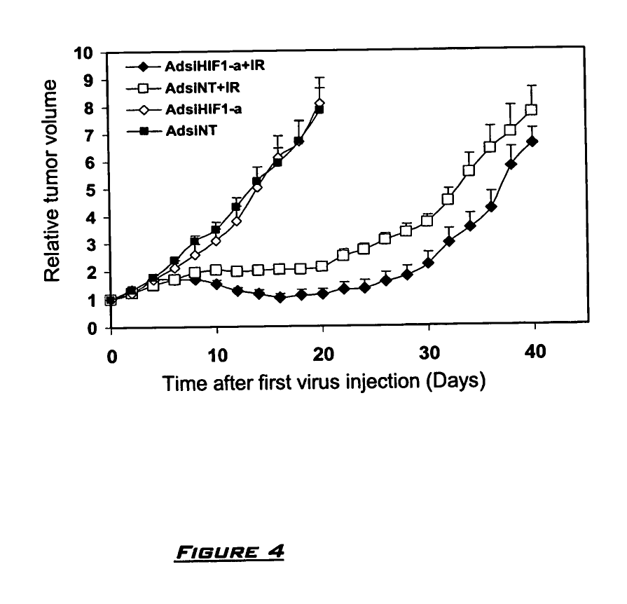

The effects of HIF-1α down regulation on tumor growth were also examined. HeLa cells or HCT116 cells infected with AdsiHIF-1α or AdsiNT (m.o.i.=10) for 24 hours were implanted subcutaneously into nude mice and tumor growth was measured. As can be seen in FIGS. 3A and 3B, both HeLa cells and HCT116 cells infected with AdsiHIF-1α grew significantly more slowly than cells infected with AdsiNT. In addition, immunohistochemistry analysis of resected tumors indicated that AdsiHIF-1α indeed suppressed the expression of the HIF-1α significantly (FIG. 3C).

Taken together, the above Examples indicate that siRNA-mediated down regulation of HIF-1α expression can effectively sensitize tumor cells to hypoxia-induced cell death. It can also significantly suppress tumor growth. As such, HIF-1α is a prime target for anticancer therapeutics development, and adenovirus-mediated delivery of siRNA is an effective approach to silence gene expression in tumor cells for gene therapy or gene function studies.

### REFERENCES

The references listed below as well as all references cited in the specification are incorporated herein by reference to the extent that they supplement, explain, provide a background for or teach methodology, techniques and/or compositions employed herein.
Adelman J P, Hayflick J S, Vasser M & Seeburg P H (1983) In Vitro Deletional Mutagenesis for Bacterial Production of the 20,000-Dalton Form of Human Pituitary Growth Hormone. DNA 2:183-193.
Alam J & Cook J L (1990) Reporter genes: application to the study of mammalian gene transcription. Anal Biochem 188:245-254.
Altschul S F, Gish W, Miller W, Myers E W & Lipman D J (1990) Basic Local Alignment Search Tool. J Mol Biol 215:403-410.
Ausubel F (ed) (1995) Short Protocols in Molecular Biology, 3rd ed. Wiley, New York, N.Y., United States of America.
Ausubel F M, Brent R. Kingston R E, Moore D D, Seidman J G, Smith J A & Struhl K, eds. (1992) Current Protocols in Molecular Biology. Wiley, New York.
Bass B L (2001) RNA interference: The short answer. Nature 411:428-429.
Beaucage & Iyer (1993) The functionalization of oligonucleotides via phosphoramidite derivative. Tetrahedron 49:1925-1963.
Beigelman L, McSwiggen J A, Draper K G, Gonzalez C, Jensen K, Karpeisky A M, Modak A S, Matulic-Adamic J, DiRenzo A B, Haeberli P, et al. (1995) Chemical modification of hammerhead ribozymes. Catalytic activity and nuclease resistance. J Biol Chem 270:25702-25708.
Bellon L, Workman C T, Jarvis T C & Wincott F E (1997) Post-synthetically ligated ribozymes: an alternative approach to iterative solid-phase synthesis. Bioconjugate Chem 8:204-212.
Bernstein E, Caudy A A, Hammond S M & Hannon G J (2001) Role for a bidentate ribonuclease in the initiation step of RNA interference. Nature 409:363-366.
Brennan T, Biddison G, Frauendorf A, Schwarcz L, Keen B, Ecker D J, Davis P W, Tinder R & Swayze E E (1998) Two-dimensional parallel array technology as a new approach to automated combinatorial solid-phase organic synthesis. Biotechnol Bioeng 61:33-45.
Brickell P M (1992) The P60c-Src Family of Protein-Tyrosine Kinases: Structure, Regulation, and Function. Crit Rev Oncog 3:401-446.
Brummelkamp T R, Bemards R & Agami R A (2002) System for stable expression of short interfering RNAs in mammalian cells. Science 296:550-553.
Burgin A B Jr, Gonzalez C, Matulic-Adamic J, Karpeisky A M, Usman N, McSwiggen J A & Beigelman L (1996) Chemically modified hammerhead ribozymes with improved catalytic rates. Biochemistry 35:14090-14097.
Burlina F, Favre A & Fourrey J L (1997) Chemical engineering of RNase resistant and catalytically active hammerhead ribozymes. Bioorg Med Chem 5:1999-2010.
Canadian Patent Application No. 2,359,180
Caruthers M H, Beaton G, Wu J V & Wiesler W (1992) Chemical synthesis of deoxyoligonucleotides and deoxyoligonucleotide analogs. Methods Enzymol 211:3-19.
Cipolla D C, Gonda I, Shak S, Kovesdi I, Crystal R & Sweeney T D (2000) Coarse Spray Delivery to a Localized Region of the Pulmonary Airways for Gene Therapy. Hum Gene Ther 11:361-371.
Clifford S C & Maher E R (2001) Von Hippel-Lindau Disease: Clinical and Molecular Perspectives. Adv Cancer Res 82:85-105.
Cubitt A B, Heim R. Adams S R, Boyd A E, Gross L A & Tsien R Y (1995) Understanding, Improving and Using Green Fluorescent Proteins. Trends Biochem Sci 20:448-455.
Dachs G U & Tozer G M (2000) Hypoxia Modulated Gene Expression: Angiogenesis, Metastasis and Therapeutic Exploitation. Eur J Cancer 36:1649-1660.
Dai S, Huang M L, Hsu C Y & Chao K S (2003) Inhibition of hypoxia inducible factor 1alpha causes oxygen-independent cytotoxicity and induces p53 independent apoptosis in glioblastoma cells. Int J Radiat Oncol Biol Phys 55:1027-36.
De Mesmaeker A, Waldner A, Lebreton J, Fritsch V & Wolf R M (1994) Novel Backbone Replacements for Oligonucleotides, in Carbohydrate Modifications in Antisense Research, American Chemical Society, Washington, D.C., Symposium Series No. 580:24-39.
Eamshaw D J & Gait M J (1998) Modified oligoribonucleotides as site-specific probes of RNA structure and function. Biopolymers 48:39-55.
Elbashir S M, Harborth J, Lendeckel W, Yalcin A, Weber K & Tuschl T (2001a) Duplexes of 21-nucleotide RNAs mediate RNA interference in cultured mammalian cells. Nature 411:494-498.
Elbashir S M, Lendeckel W & Tuschl T (2001b) RNA interference is mediated by 21- and 22-nucleotide RNAs. Genes Dev 15:188-200.
Elbashir S M, Martinez J. Patkaniowska A, Lendeckel W & Tuschl T (2001c) Functional anatomy of siRNAs for mediating efficient RNAi in Drosophila melanogaster embryo lysate. EMBO J. 20:6877-88.
European Patent No. 0 439 095
Fire A, Xu S, Montgomery M K, Kostas S A, Driver S E & Mello C C (1998) Chromatin silencing and the maintenance of a functional germline in Caenorhabditis elegans. Nature 391:806-811.
Fire A (1999) RNA-triggered gene silencing. Trends Genet 15:358-363.
Freier S M, Kierzek R, Jaeger J A, Sugimoto N, Caruthers M H, Neilson T & Turner D H (1986) Improved Free-Energy Parameters for Predictions of RNA Duplex Stability. Proc Natl Acad Sci USA 83:9373-9377.
Glover D M & Hames B D (1995) DNA Cloning: A Practical Approach, 2nd ed. IRL Press at Oxford University Press, Oxford; New York.
Greenberg N M, DeMayo F J, Sheppard P C, Barrios R, Lebovitz R, Finegold M, Angelopoulou R, Dodd J G, Duckworth M L, Rosen J M et al. (1994) The Rat Probasin Gene Promoter Directs Hormonally and Developmentally Regulated Expression of a Heterologous Gene Specifically to the Prostate in Transgenic Mice. Mol Endocrinol 8:230-239.
Habib N A, Hodgson H J, Lemoine N & Pignatelli M (1999) A Phase I/Ii Study of Hepatic Artery Infusion with wtp53-CMV-Ad in Metastatic Malignant Liver Tumours. Hum Gene Ther 10:2019-2034.
Hammond S M, Bernstein E, Beach D & Hannon G J (2000) An RNA-directed nuclease mediates post-transcriptional gene silencing in Drosophila cells. Nature 404:293-296.
He T C, Zhou S, da Costa L T, Yu J, Kinzler K W & Vogelstein B A (1998) Simplified system for generating recombinant adenoviruses. Proc Natl Acad Sci USA 95:2509-2514.
Henikoff S & Henikoff J G (1992) Amino Acid Substitution Matrices from Protein Blocks. Proc Natl Acad Sci U SA 89:10915-10919.
Hunziker J & Leumann C (1995) Nucleic Acid Analogues: Synthesis and Properties, in Modern Synthetic Methods, VCH, Basel, Switzerland 331-417.
Ivan M & Kaelin W G, Jr. (2001) The Von Hippel-Lindau Tumor Suppressor Protein. Curr Opin Genet Dev 11:27-34.
Karlin S & Altschul S F (1993) Applications and Statistics for Multiple High-Scoring Segments in Molecular Sequences. Proc Natl Acad Sci USA 90:5873-5877.
Karni R, Dor Y, Keshet E, Meyuhas O & Levitzki A (2002) Activated Pp60c-Src Leads to Elevated HIF-1 Alpha Expression under Normoxia. J Biol Chem: M206141200.
Karpeisky A, Gonzales C, Burgin A B & Beigelman, L (1998) Highly efficient synthesis of 2′-O-amino nucleosides and their incorporation in hammerhead ribozymes. Tetrahedron Lett 39:1131-1134.
Kurihara T, Brough D E, Kovesdi I & Kufe D W (2000) Selectivity of a Replication competent Adenovirus for Human Breast Carcinoma Cells Expressing the MUC1 Antigen. J Clin Invest 106:763-771.
Lee S E, Jin R J, Lee S G, Yoon S J, Park M S, Heo D S & Choi H (2000) Development of a New Plasmid Vector with PSA-Promoter and Enhancer Expressing Tissue-Specificity in Prostate Carcinoma Cell Lines. Anticancer Res 20:417-422.
Limbach P A, Crain P F & McCloskey J A (1994) Summary: the modified nucleosides of RNA. Nucleic Acids Res 22:2183-.
Lindegaard J C, Overgaard J. Bentzen S M & Pedersen D (1996) Is There a Radiobiologic Basis for Improving the Treatment of Advanced Stage Cervical Cancer? J Natl Cancer Inst Monogr21:105-112.
Loakes D (2001) Survey and summary: The applications of universal DNA base analogues. Nucleic Acids Res 29:2437-2447.
Maxwell P H, Pugh C W & Ratcliffe P J (2001) Activation of the HIF Pathway in Cancer. Curr Opin Genet Dev 11:293-299.
Miyagishi M & Taira K. (2002) U6 promoter-driven siRNAs with four uridine 3′ overhangs efficiently suppress targeted gene expression in mammalian cells. Nat Biotechnol 20:497-500.
Needleman S B & Wunsch C D (1970) A General Method Applicable to the Search for Similarities in the Amino Acid Sequence of Two Proteins. J Mol Biol 48:443-453.
Nykanen A, Haley B & Zamore P D (2001) ATP requirements and small interfering RNA structure in the RNA interference pathway. Cell 107:309-321.
PCT International Publication No. WO 91/03162
PCT International Publication No. WO 92/07065
PCT International Publication No. WO 93/15187
PCT International Publication No. WO 93/23569
PCT International Publication No. WO 96/33280
PCT International Publication No. WO 97/26270
PCT International Publication No. WO 97/45550
PCT International Publication No. WO 97/47763
PCT International Publication No. WO 98/13526
PCT International Publication No. WO 98/54345
PCT International Publication No. WO 99/07409
PCT International Publication No. WO 99/32619
PCT International Publication No. WO 99/54459
PCT International Publication No. WO 00/01846
PCT International Publication No. WO 00/44895
PCT International Publication No. WO 00/44914
PCT International Publication No. WO 00/63364
PCT International Publication No. WO 01/04313
PCT International Publication No. WO 01/29058
PCT International Publication No. WO 01/36646
PCT International Publication No. WO 01/68836
PCT International Publication No. WO 01/75164
PCT International Publication No. WO 01/92513
PCT International Publication No. WO 02/044321
PCT International Publication No. WO 02/055692
PCT International Publication No. WO 02/055693
Pearson W R & Lipman D J (1988) Improved Tools for Biological Sequence Comparison. Proc Natl Acad Sci USA 85:2444-2448.
Perrault et al. (1990) Nature 344:565.
Pieken W A, Olsen D B, Benseler F, Aurup H & Eckstein F (1991) Kinetic characterization of ribonuclease-resistant 2′-modified hammerhead ribozymes. Science 253:314-317.
Rose M & Botstein D (1983) Construction and use of gene fusions to lacZ (betagalactosidase) that are expressed in yeast. Meth Enzymol 101:167-180.
Rothmann T, Hengstermann A, Whitaker N J, Scheffner M & zur Hausen H (1998) Replication of Onyx-015, a Potential Anticancer Adenovirus, Is Independent of p53 Status in Tumor Cells. J Virol 72:9470-9478.
Sambrook J & Russell D W (2001) Molecular Cloning: A Laboratory Manual, 3rd ed. Cold Spring Harbor Laboratory Press, Cold Spring Harbor, N.Y.
Scaringe S A, Francklyn C & Usman N (1990) Chemical synthesis of biologically active oligoribonucleotides using beta-cyanoethyl protected ribonucleoside phosphoramidites. Nucleic Acids Res 18:5433-5441.
Scharfmann R, Axelrod J H & Verma I M (1991) Long-Term in Vivo Expression of Retrovirus-Mediated Gene Transfer in Mouse Fibroblast Implants. Proc Natl Acad Sci USA 88:4626-4630.
Semenza G L, Nejfelt M K, Chi S M & Antonarakis S E (1991) Hypoxia inducible Nuclear Factors Bind to an Enhancer Element Located 3′ to the Human Erythropoietin Gene. Proc Natl Acad Sci USA 88:5680-5684.
Shabarova Z A, Merenkova I N, Oretskaya T S, Sokolova N I, Skripkin E A, Alexeyeva E V, Balakin A G & Bogdanov M (1991) Chemical ligation of DNA: the first non-enzymatic assembly of a biologically active gene. Nucleic Acids Res 19:4247-4251.
Silhavy T J, Berman M L, Enquist L W & Cold Spring Harbor Laboratory. (1984) Experiments with Gene Fusions. Cold Spring Harbor Laboratory, Cold Spring Harbor, N.Y.
Sinkovics J G & Horvath J C (2000) Vaccination against Human Cancers (Review). Int J Oncol 16:81-96.
Smith T F & Waterman M (1981) Comparison of Biosequences. Adv Appl Math 2:482-489.
Sowter H M, Ratcliffe P J, Watson P, Greenberg A H & Harris A L (2001) HIF-1-dependent regulation of hypoxic induction of the cell death factors BNIP3 and NIX in human tumors. Cancer Res 61:6669-6673.
Suit H (1996) Assessment of the Impact of Local Control on Clinical Outcome. Front Radiat Ther Oncol 29:17-23.
Tijssen P (1993) Laboratory Techniques in Biochemistry and Molecular Biology-Hybridization with Nucleic Acid Probes. Elsevier, New York, United States of America.
Turner D H, Sugimoto N, Jaeger J A, Longfellow C E, Freier S M & Kierzek R (1987) Improved parameters for prediction of RNA structure. Cold Spring Harb Symp Quant Biol LII:123-133.
Uhlman E & Peyman A (1990) Antisense oligonucleotides: a new therapeutic principle. Chem Rev 90:543-549.
Usman N, Beigelman L, Draper K, Gonzalez C, Jensen K, Karpeisky A, Modak A, Matulic-Adamic J. DiRenzo A, Haeberli P, Tracz D, Grimm S, Wincott F & McSwiggen J (1994) Nucleic Acids Symp Ser 31:163-164.
Usman N, Beigelman L & McSwiggen J A (1996) Hammerhead ribozyme engineering. Curr Opin Struct Biol 6:527-33.
Usman N & Cedergren R (1992) Exploiting the chemical synthesis of RNA. Trends Biochem Sci 17:334-339.
Usman N, Ogilvie K K, Jiang M Y & Cedergren R J (1987) Automated chemical synthesis of long oligoribonucleotides using 2′-O-silylated ribonucleoside 3′-O-phosphoramidites on a controlled-pore glass support—synthesis of a 43-nucleotide sequence similar to the 3′-half molecule of an Escherichia coli formylmethionine transfer-RNA J Am Chem Soc 109: 7845-7854.
U.S. Pat. No. 4,554,101
U.S. Pat. No. 5,334,711
U.S. Pat. No. 5,627,053
U.S. Pat. No. 5,672,695
U.S. Pat. No. 5,716,824
U.S. Pat. No. 5,854,038
U.S. Pat. No. 5,858,784
U.S. Pat. No. 5,871,982
U.S. Pat. No. 5,998,203
U.S. Pat. No. 6,001,311
U.S. Pat. No. 6,013,638
U.S. Pat. No. 6,022,737
U.S. Pat. No. 6,136,295
U.S. Pat. No. 6,248,878
U.S. Pat. No. 6,300,074
Valter M M, Hugel A, Huang H J, Cavenee W K, Wiestler O D, Pietsch T & Wernert N (1999) Expression of the Ets-1 Transcription Factor in Human Astrocytomas Is Associated with Fms-Like Tyrosine Kinase-1 (Flt-1)/Vascular Endothelial Growth Factor Receptor-1 Synthesis and Neoangiogenesis. Cancer Res 59:5608-5614.
Verma S & Eckstein F (1998) Modified oligonucleotides: synthesis and strategy for users. Annu Rev Biochem 67:99-134.
Vose J M & Armitage J O (1995) Clinical Applications of Hematopoietic Growth Factors. J Clin Oncol 13:1023-1035.
Wianny F & Zernicka-Goetz M (1999) Specific interference with gene function by double-stranded RNA in early mouse development. Nature Cell Biol 2:70-75.
Williams R S, Thomas J A, Fina M, German Z & Benjamin I J (1993) Human Heat Shock Protein 70 (Hsp70) Protects Murine Cells from Injury During Metabolic Stress. J Clin Invest 92:503-508.
Wincott F, DiRenzo A, Shaffer C, Grimm S, Tracz D, Workman C, Sweedler D, Gonzalez C, Scaringe S & Usman N (1995) Synthesis, deprotection, analysis and purification of RNA and ribozymes. Nucleic Acids Res 23:2677-2684.
Wincott F E & Usman N (1997) A practical method for the production of RNA and ribozymes. Methods Mol Bio 74: 59-68.
Yazawa K, Fisher W E & Brunicardi F C (2002) Current Progress in Suicide Gene Therapy for Cancer. World J Surg 26:783-789.
Yu D C, Chen Y. Seng M, Dilley J & Henderson DR (1999) The Addition of Adenovirus Type 5 Region E3 Enables Calydon Virus 787 to Eliminate Distant Prostate Tumor Xenografts. Cancer Res 59:4200-4203.
Yu J Y, DeRuiter S L & Turner D L (2002) RNA interference by expression of short-interfering RNAs and hairpin RNAs in mammalian cells. Proc Natl Acad Sci USA 99:6047-6052.
Zhang X, Li Y, Huang Q, Wang H, Yan B, Dewhirst M W & Li C Y (2003) Increased Resistance of Tumor Cells to Hyperthermia Mediated By Integrin-linked Kinase. Clin Cancer Res 9:1155-1160.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method for inhibiting expression of a hypoxia-inducible gene in a cell in hypoxic conditions or expected to undergo hypoxic conditions, the method comprising introducing a ribonucleic acid (RNA) into the cell in an amount sufficient to inhibit expression of the hypoxia-inducible gene, wherein the RNA comprises a ribonucleotide sequence which corresponds to a coding strand of the hypoxia-inducible gene.

2. The method of claim 1, wherein the hypoxia-inducible gene is HIF-1α.

3. The method of claim 2, wherein the HIF-1α gene comprises a nucleotide sequence of one of SEQ ID NOs: 1 and 3.

4. The method of claim 1, wherein the RNA comprises a double-stranded region comprising a first strand comprising a ribonucleotide sequence that corresponds to a coding strand of the hypoxia-inducible gene and a second strand comprising a ribonucleotide sequence that is complementary to the first strand, and wherein the first strand and the second strand hybridize to each other to form the double-stranded molecule.

5. The method of claim 4, wherein the RNA comprises one strand that forms a double-stranded region by intramolecular self-hybridization that is complementary over at least 19 bases.

6. The method of claim 4, wherein the RNA comprises two separate strands that form a double-stranded region by intermolecular hybridization that is complementary over at least 19 bases.

7. The method of claim 4, wherein the double stranded region is at least 15 basepairs in length.

8. The method of claim 7, wherein the double stranded region is between 15 and 50 basepairs in length.

9. The method of claim 8, wherein the double stranded region is between 19 and 30 basepairs in length.

10. The method of claim 4, wherein a length of the double stranded region is selected from the group consisting of 19, 20, 21, 22, 23, 24, 25, 26, 27, 28, 29, and 30 basepairs.

11. The method of claim 1, wherein the expression of the hypoxia-inducible gene is inhibited by at least 10%.

12. The method of claim 1, wherein the cell is present in an organism, and the RNA is introduced into the organism.

13. The method of claim 1, wherein the cell is present in an organism and the RNA is introduced by extracellular injection into the organism.

14. The method of claim 1, further comprising introducing a vector into the cell, wherein the vector encodes the RNA.

15. A method for inhibiting expression of a hypoxia-inducible gene in a subject, the method comprising:
(a) providing a subject containing a target cell, wherein the target cell comprises the hypoxia-inducible gene and the hypoxia-inducible gene is expressed in the target cell when the target cell is exposed to hypoxic conditions; and
(b) introducing a small interfering RNA (siRNA) into the target cell, wherein the siRNA comprises a nucleic acid sequence corresponding to the hypoxia-inducible gene.

16. The method of claim 15, wherein the subject is an animal.

17. The method of claim 15, wherein the small interfering RNA (siRNA) comprises a double-stranded structure with duplexed ribonucleic acid strands and one of the strands is complementary to a portion of the hypoxia-inducible gene.

18. The method of claim 15 wherein the small interfering RNA (siRNA) is introduced into the subject and outside the target cell.

19. The method of claim 15, wherein the introducing a small interfering RNA (siRNA) into the target cell comprises introducing a vector encoding the small interfering RNA (siRNA) into the target cell.

20. A method for suppressing the growth of a hypoxic cell in a subject, the method comprising contacting the cell with a vector comprising a small interfering RNA (siRNA) molecule under conditions sufficient to allow entry of the vector into the cell, wherein the siRNA molecule comprises a sense region and an antisense region and wherein the antisense region comprises a nucleic acid sequence complementary to an RNA sequence encoding a hypoxia-inducible gene product and the sense region comprises a nucleic acid sequence complementary to the antisense region.

21. The method of claim 20, wherein the cell is a tumor cell.

22. The method of claim 21, wherein the tumor cell is in a hypoxic region of a tumor.

23. The method of claim 22, wherein the subject is a mammal.

24. The method of claim 20, wherein the vector comprises a liposome.

25. The method of claim 20, wherein the hypoxia-inducible gene is hypoxia inducible factor 1 alpha (HIF-1α).

26. The method of claim 25, wherein the HIF-1α gene comprises a nucleotide sequence of one of SEQ ID NOs: 1 and 3.

27. The method of claim 20, wherein the introducing is via a route of administration selected from the group consisting of intravenous administration, intrasynovial administration, transdermal administration, intramuscular administration, subcutaneous administration, topical administration, rectal administration, intravaginal administration, intratumoral administration, oral administration, buccal administration, nasal administration, parenteral administration, inhalation, and insufflation.

28. A method for suppressing the growth of a hypoxic cell in a subject, the method comprising contacting the cell with a vector encoding a small interfering RNA (siRNA) molecule under conditions sufficient to allow entry of the vector into the cell, wherein the siRNA molecule comprises a sense region and an antisense region and wherein the antisense region comprises a first nucleic acid sequence that is 100% complementary to at least 19 contiguous nucleotides of a hypoxia-inducible gene sequence and the sense region comprises a second nucleic acid sequence that is 100% complementary to the first nucleic acid sequence.

29. The method of claim 28, wherein the cell is a tumor cell.

30. The method of claim 29, wherein the tumor cell is in a hypoxic region of a tumor.

31. The method of claim 28, wherein the subject is a mammal.

32. The method of claim 28, wherein the vector is an adenovirus vector.

33. The method of claim 28, wherein the hypoxia-inducible gene is hypoxia inducible factor 1 alpha (HIF-1α).

34. The method of claim 33, wherein the HIF-1α gene comprises a nucleotide sequence of one of SEQ ID NOs: 1 and 3.

35. The method of claim 28, wherein the introducing is via a route of administration selected from the group consisting of intravenous administration, intrasynovial administration, transdermal administration, intramuscular administration, subcutaneous administration, topical administration, rectal administration, intravaginal administration, intratumoral administration, oral administration, buccal administration, nasal administration, parenteral administration, inhalation, and insufflation.

36. A small interfering RNA (siRNA) molecule that down regulates expression of a hypoxia-inducible factor 1α (HIF-1α) gene by RNA interference.

37. The siRNA molecule of claim 36, wherein the siRNA molecule comprises a sense region and an antisense region and wherein the antisense region comprises a first nucleic acid sequence that is 100% complementary to at least 19 contiguous nucleotides of a hypoxia-inducible factor 1α (HIF1-α) gene sequence and the sense region comprises a second nucleic acid sequence that is 100% complementary to the first nucleic acid sequence.

38. The siRNA molecule of claim 37, wherein the siRNA molecule is assembled from two nucleic acid fragments, wherein one fragment comprises a sense region and the other fragment comprises an antisense region of the siRNA molecule.

39. The siRNA molecule of claim 38, wherein the sense region and antisense region are covalently connected via a linker molecule.

40. The siRNA molecule of claim 39, wherein the linker molecule is a polynucleotide linker.

41. The siRNA molecule of claim 40, wherein the polynucleotide linker comprises from 5 to 9 nucleotides.

42. The siRNA molecule of claim 39, wherein the linker molecule is a non-nucleotide linker.

43. The siRNA molecule of claim 37, wherein the sense region comprises a contiguous 19-30 nucleotide subsequence of one of SEQ ID NOs. 1 and 3.

44. The siRNA molecule of claim 37, wherein the sense region comprises the sequence 5′-GATGACATGAAAGCACAGA-3′ (SEQ ID NO: 7) and the antisense region comprises a 100% reverse-complement of SEQ ID NO: 7.

45. The siRNA molecule of claim 37, wherein at least one of the sense region and the antisense region comprises a 3′-terminal overhang.

46. The siRNA molecule of claim 45, wherein at least one 3′-terminal overhang comprises from 2 to 8 nucleotides.

47. The siRNA molecule of claim 37, wherein the sense region comprises one or more modified pyrimidine nucleotides.

48. The siRNA molecule of claim 37, wherein the sense region comprises a terminal cap moiety at a 5′-end, a 3′-end, or combinations thereof.

49. The siRNA molecule of claim 37, wherein the antisense region comprises one or more modified pyrimidine nucleotides.

50. The siRNA molecule of claim 37, wherein the antisense region comprises a phosphorothioate internucleotide linkage at a 3′ end of the antisense region.

51. The siRNA molecule of claim 37, wherein the antisense region comprises 1-5 phosphorothioate internucleotide linkages at a 5′ end of the antisense region.

52. The siRNA molecule of claim 45, wherein the 3′-terminal nucleotide overhang comprises one or more chemically modified nucleotides.

53. The siRNA molecule of claim 52, wherein the 3′-terminal nucleotide overhang comprises ribonucleotides that are chemically modified at a nucleic acid sugar, base, or backbone position.

54. The siRNA molecule of claim 45, wherein the 3′-terminal nucleotide overhang comprises one or more universal base ribonucleotides.

55. The siRNA molecule of claim 45, wherein the 3′-terminal nucleotide overhang comprises one or more acyclic nucleotides.

56. The siRNA molecule of claim 45, in a pharmaceutically acceptable carrier.

57. An expression vector comprising a nucleic acid sequence encoding at least one siRNA molecule of claim 36.

58. A mammalian cell comprising an expression vector of claim 57.

59. The mammalian cell of claim 58, wherein the mammalian cell is a human cell.

60. The expression vector of claim 57, wherein the siRNA molecule comprises a sense region and an antisense region and wherein the antisense region comprises a first nucleic acid sequence that is 100% complementary to at least 19 contiguous nucleotides of a hypoxia-inducible factor 1α (HIF-1α) gene sequence and the sense region comprises a second nucleic acid sequence that is 100% complementary to the first nucleic acid sequence.

61. The expression vector of claim 57, wherein the siRNA molecule comprises two distinct strands having complementary sense and antisense regions.

62. The expression vector of claim 57, wherein the siRNA molecule comprises a single strand having complementary sense and antisense regions.

63. An adenovirus vector comprising the small interfering RNA (siRNA) molecule of claim 36.


SEQUENCE LISTING (12 sequences)

SEQ ID NO: 1 - DNA, 3958 bp, Homo sapiens
  CDS (285)..(2765)
gtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcactacctaaagcagtctatttatattttctacatctaattttagaagcctggctacaatactgcacaaacttggttagttcaatttttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccaggaaaaaaaaaaaaaaaaaaa

SEQ ID NO: 2 - protein, 826 aa, Homo sapiens
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 3 - DNA, 3973 bp, Mus musculus
  CDS (258)..(2768)
cgcgaggactgtcctcgccgccgtcgcgggcagtgtctagccaggccttgacaagctagccggaggagcgcctaggaacccgagccggagctcagcgagcgcagcctgcacgcccgcctcgcgtcccgggggggtcccgcctcccaccccgcctctggacttgtctctttccccgcgcgcgcggacagagccggcgtttaggcccgagcgagcccgggggccgccggccgggaagacaacgcgggcaccgattcgccatggagggcgccggcggcgagaacgagaagaaaaagatgagttctgaacgtcgaaaagaaaagtctagagatgcagcaagatctcggcgaagcaaagagtctgaagttttttatgagcttgctcatcagttgccacttccccacaatgtgagctcacatcttgataaagcttctgttatgaggctcaccatcagttatttacgtgtgagaaaacttctggatgccggtggtctagacagtgaagatgagatgaaggcacagatggactgtttttatctgaaagccctagatggctttgtgatggtgctaacagatgacggcgacatggtttacatttctgataacgtgaacaaatacatggggttaactcagtttgaactaactggacacagtgtgtttgattttactcatccatgtgaccatgaggaaatgagagaaatgcttacacacagaaatggcccagtgagaaaagggaaagaactaaacacacagcggagcttttttctcagaatgaagtgcaccctaacaagccgggggaggacgatgaacatcaagtcagcaacgtggaaggtgcttcactgcacgggccatattcatgtctatgataccaacagtaaccaacctcagtgtgggtacaagaaaccacccatgacgtgcttggtgctgatttgtgaacccattcctcatccgtcaaatattgaaattcctttagatagcaagacatttctcagtcgacacagcctcgatatgaaattttcttactgtgatgaaagaattactgagttgatgggttatgagccggaagaacttttgggccgctcaatttatgaatattatcatgctttggattctgatcatctgaccaaaactcaccatgatatgtttactaaaggacaagtcaccacaggacagtacaggatgcttgccaaaagaggtggatatgtctgggttgaaactcaagcaactgtcatatataatacgaagaactcccagccacagtgcattgtgtgtgtgaattatgttgtaagtggtattattcagcacgacttgattttctcccttcaacaaacagaatctgtgctcaaaccagttgaatcttcagatatgaagatgactcagctgttcaccaaagttgaatcagaggatacaagctgcctttttgataagcttaagaaggagcctgatgctctcactctgctggctccagctgccggcgacaccatcatctctctggattttggcagcgatgacacagaaactgaagatcaacaacttgaagatgttccattatataatgatgtaatgtttccctcttctaatgaaaaattaaatataaacctggcaatgtctcctttaccttcatcggaaactccaaagccacttcgaagtagtgctgatcctgcactgaatcaagaggttgcattaaaattagaatcaagtccagagtcactgggactttcttttaccatgccccagattcaagatcagccagcaagtccttctgatggaagcactagacaaagttcacctgagagacttcttcaggaaaacgtaaacactcctaacttttcccagcctaacagtcccagtgaatattgctttgatgtggatagcgatatggtcaatgtattcaagttggaactggtggaaaaactgtttgctgaagacacagaggcaaagaatccattttcaactcaggacactgatttagatttggagatgctggctccctatatcccaatggatgatgatttccagttacgttcctttgatcagttgtcaccattagagagcaattctccaagccctccaagtatgagcacagttactgggttccagcagacccagttacagaaacctaccatcactgccactgccaccacaactgccaccactgatgaatcaaaaacagagacgaaggacaataaagaagatattaaaatactgattgcatctccatcttctacccaagtacctcaagaaacgaccactgctaaggcatcagcatacagtggcactcacagtcggacagcctcaccagacagagcaggaaagagagtcatagaacagacagacaaagctcatccaaggagccttaagctgtctgccactttgaatcaaagaaatactgttcctgaggaagaattaaacccaaagacaatagcttcgcagaatgctcagaggaagcgaaaaatggaacatgatggctccctttttcaagcagcaggaattggaacattattgcagcaaccaggtgactgtgcacctactatgtcactttcctggaaacgagtgaaaggattcatatctagtgaacagaatggaacggagcaaaagactattattttaataccctccgatttagcatgcagactgctggggcagtcaatggatgagagtggattaccacagctgaccagttacgattgtgaagttaatgctcccatacaaggcagcagaaacctactgcagggtgaagaattactcagagctttggatcaagttaactgagcgtttcctaatctcattccttttgattgttaatgtttttgttcagttgttgttgtttgttgggtttttgtttctgttggttatttttggacactggtggctcagcagtctatttatattttctatatctaattttagaagcctggctacaatactgcacaaactcagatagtttagttttcatcccctttctacttaattttcattaatgctctttttaatatgttcttttaatgccagatcacagcacattcacagctcctcagcatttcaccattgcattgctgtagtgtcatttaaaatgcacctttttatttatttatttttggtgagggagtttgtcccttattgaattatttttaatgaaatgccaatataattttttaagaaagcagtaaattctcatcatgatcataggcagttgaaaactttttactcatttttttcatgttttacatgaaaataatgctttgtcagcagtacatggtagccacaattgcacaatatattttctttaaaaaaccagcagttactcatgcaatatattctgcatttataaaactagtttttaagaaattttttttggcctatggaattgttaagcctggatcatgaagcgttgatcttataatgattcttaaactgtatggtttctttatatgggtaaagccatttacatgatataaagaaatatgcttatatctggaaggtatgtggcatttatttggataaaattctcaattcagagaagttatctggtgtttcttgactttaccaactcaaaacagtccctctgtagttgtggaagcttatgctaatattgtgtaattgattatgaaacataaatgttctgcccaccctgttggtataaagacattttgagcatactgtaaacaaacaaacaaaaaatcatgctttgttagtaaaattgcctagtatgttgatttgttgaaaatatgatgtttggttttatgcactttgtcgctattaacatccttttttcatatagatttcaataagtgagtaattttagaagcattattttaggaatatagagttgtcatagtaaacatcttgttttttctatgtacactgtataaatttttcgttcccttgctctttgtggttgggtctaacactaactgtactgttttgttatatcaaataaacatcttctgtggaccaggaaaaaaaaaaaaaaaaaa

SEQ ID NO: 4 - protein, 836 aa, Mus musculus
MEGAGGENEKKKMSSERRKEKSRDAARSRRSKESEVFYELAHQLPLPHNVSSHLDKASVMRLTISYLRVRKLLDAGGLDSEDEMKAQMDCFYLKALDGFVMVLTDDGDMVYISDNVNKYMGLTQFELTGHSVFDFTHPCDHEEMREMLTHRNGPVRKGKELNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTESVLKPVESSDMKMTQLFTKVESEDTSCLFDKLKKEPDALTLLAPAAGDTIISLDFGSDDTETEDQQLEDVPLYNDVMFPSSNEKLNINLAMSPLPSSETPKPLRSSADPALNQEVALKLESSPESLGLSFTMPQIQDQPASPSDGSTRQSSPERLLQENVNTPNFSQPNSPSEYCFDVDSDMVNVFKLELVEKLFAEDTEAKNPFSTQDTDLDLEMLAPYIPMDDDFQLRSFDQLSPLESNSPSPPSMSTVTGFQQTQLQKPTITATATTTATTDESKTETKDNKEDIKILIASPSSTQVPQETTTAKASAYSGTHSRTASPDRAGKRVIEQTDKAHPRSLKLSATLNQRNTVPEEELNPKTIASQNAQRKRKMEHDGSLFQAAGIGTLLQQPGDCAPTMSLSWKRVKGFISSEQNGTEQKTIILIPSDLACRLLGQSMDESGLPQLTSYDCEVNAPIQGSRNLLQGEELLRALDQVN

SEQ ID NO: 5 - DNA, 55 bp, Artificial
  Generic sequence of an siRNA used to target human HIF-1a
  misc_feature (20)..(28): n can be any nucleotide, up to 4 of which can be missing, representing a single stranded loop of from 5-9 bases, the hairpin loop remaining single-stranded when bases 1-19 and 29-47 hybridize to each other to form a duplex
  misc_feature (48)..(55): n can be any nucleotide, up to 6 of which can be missing, representing a 3′ overhang of from 2-8 nucleotides, the 3′ overhang remaining single stranded when the duplex forms between nucleotides 1-19 and 29-47
gatgacatgaaagcacagannnnnnnnntctgtgctttcatgtcatcnnnnnnnn

SEQ ID NO: 6 - DNA, 53 bp, Artificial
  Specific sequence of an siRNA used to target human HIF-1a
  misc_feature (1)..(19): Sense strand of an siRNA used to target human HIF-1a. Sequence corresponds to bases 528-546 of Genbank Accession No. NM_001530. This sequence forms the double stranded region of a hairpin by intramolecular hybridization with bases 29-47.
  misc_feature (20)..(28): 9 base loop structure, whichs stays single stranded when bases 1-19 and 29-47 form a duplex
  misc_feature (29)..(47): Antisense strand of an siRNA used to target human HIF-1a. Sequence corresponds to the reverse complement of bases 528-546 of NM_001530. This sequence forms the double stranded region of a hairpin by intramolecular hybridization with bases 1-19.
  misc_feature (48)..(53): 6 base 3′ overhang, which stays single stranded when bases 1-19 and 29-47 form a duplex
gatgacatgaaagcacagattcaagagatctgtgctttcatgtcatctttttt

SEQ ID NO: 7 - DNA, 19 bp, Homo sapiens
atgacatgaaagcacagat

SEQ ID NO: 8 - DNA, 21 bp, Artificial
  Artificial sense strand designed with no known homology to any human gene to be used to create a negative control siRNA
aattctccgaacgtgtcacgt

SEQ ID NO: 9 - DNA, 21 bp, Homo sapiens
tcaagatcattgctcctcctg

SEQ ID NO: 10 - DNA, 21 bp, Homo sapiens
ctgcttgctgatccacatctg

SEQ ID NO: 11 - DNA, 21 bp, Homo sapiens
ctgatcatctgaccaaaactc

SEQ ID NO: 12 - DNA, 22 bp, Homo sapiens
gtttcaacccagacatatccac